(19) United States
(12) Patent Application Publication
(10) Pub. No.: US 2024/0112817 A1
(43) Pub. Date: Apr. 4, 2024

(54) REAGENT SELECTOR

(71) Applicant: Scinapsis Analytics Inc., dba BenchSci, Toronto (CA)

(72) Inventors: Casandra Savitri MANGROO, Pickering (CA); Elvis WIANDA, Oakville (CA); Tom LEUNG, North York (CA); Matan BERSON, Montreal (CA)

(73) Assignee: Scinapsis Analytics Inc., dba BenchSci, Toronto (CA)

(21) Appl. No.: 17/958,164

(22) Filed: Sep. 30, 2022

(51) Int. Cl. G16H 50/70; G06F 16/538

(52) U.S. Cl. CPC G16H 50/70; G06F 16/538

(57) ABSTRACT

A method implements reagent selection. The method includes receiving a search request comprising an entity type and an entity name; searching biomedical data to identify a set of biomedical sources that correspond to the entity name using an ontology library; and presenting an image from a biomedical source, of the set of biomedical sources, wherein the image corresponds to the entity name.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of biomedical information provide access to the electronic forms of the publications. A challenge is for computing systems to identify and display correspondences between reagents (also referred to as biomedical products) and the biomedical information describing the use of the biomedical products.

### SUMMARY

In general, in one or more aspects, the disclosure relates to a method implementing reagent selection. The method includes receiving a search request comprising an entity type and an entity name; searching biomedical data to identify a set of biomedical sources that correspond to the entity name using an ontology library; and presenting an image from a biomedical source, of the set of biomedical sources, wherein the image corresponds to the entity name.

In general, in one or more aspects, the disclosure relates to a system implementing reagent selection. The system includes an information controller configured to receive a search request; a result controller configured to search biomedical data; and an application executing on one or more processors. The application is configured for receiving, by the information controller, the search request comprising an entity type and an entity name; searching, by the result controller, the biomedical data to identify a set of biomedical sources that correspond to the entity name using an ontology library; and presenting an image from a biomedical source, of the set of biomedical sources, wherein the image corresponds to the entity name.

In general, in one or more aspects, the disclosure relates to a non-transitory computer-readable medium storing program instructions that, when executed by one or more processors, implement reagent selection. The instructions cause the computing system to perform operations including receiving a search request comprising an entity type and an entity name; searching biomedical data to identify a set of biomedical sources that correspond to the entity name using an ontology library; and presenting an image from a biomedical source, of the set of biomedical sources, wherein the image corresponds to the entity name.

Other aspects of the invention will be apparent from the following description and the appended claims.

### BRIEF DESCRIPTION OF DRAWINGS

FIG. 1A and FIG. 1B show diagrams of systems in accordance with disclosed embodiments.

FIG. 2 shows a flowchart in accordance with disclosed embodiments.

FIG. 3A, FIG. 3B, FIG. 3C, FIG. 4A, FIG. 4B, FIG. 5, FIG. 6A, FIG. 6B, FIG. 6C, FIG. 6D, FIG. 6E, FIG. 6F, FIG. 6G, FIG. 7A, FIG. 7B, FIG. 7C, FIG. 7D, FIG. 7E, FIG. 7F, FIG. 8A, FIG. 8B, FIG. 8C, FIG. 8D, FIG. 8E, FIG. 8F, FIG. 9A, FIG. 9B, FIG. 10A, FIG. 10B, FIG. 11A, FIG. 11B, FIG. 11C, FIG. 11D, FIG. 11E, FIG. 12A, FIG. 12B, and FIG. 12C show examples in accordance with disclosed embodiments.

FIG. 13A and FIG. 13B show computing systems in accordance with disclosed embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure select reagents that are described in biomedical information sources. The system processes the biomedical information sources to generate an ontology library that includes entity records. The entity records define the types and names of entities from the biomedical information sources. The types of biomedical entities include proteins, genes, diseases, experiment techniques, chemicals, cell lines, pathways, tissues, cell types, organisms, etc. The system receives selections of the types and names of entities and searches the entity records suing the types and names. Results from searching are presented in a product view or a figure view from which additional information about the entities may be presented.

In general, embodiments of the disclosure architect experiments. Users interact with the system to provide information describing experiments. The system searches evidence result graphs generated from biomedical information to the information provided by users to provide biomedical information relevant to experiments of the users.

Machine learning models are used by the system to generate the entity records from the biomedical information sources. The machine learning models may be trained to understand evidence both written and visual. For example, a machine learning model may be trained to recognize and tag entities in biomedical information that appear in a sentence. Additional machine learning models (semantic tree generators, image recognizers, etc.) may be trained with biomedical data (text and images) to be customized for biomedical data.

The figures show diagrams of embodiments that are in accordance with the disclosure. The embodiments of the figures may be combined and may include or be included within the features and embodiments described in the other figures of the application. The features and elements of the figures are, individually and as a combination, improvements to the technology of biomedical information processing and machine learning models. The various elements, systems, components, and steps shown in the figures may be omitted, repeated, combined, and/or altered as shown from the figures. Accordingly, the scope of the present disclosure should not be considered limited to the specific arrangements shown in the figures.

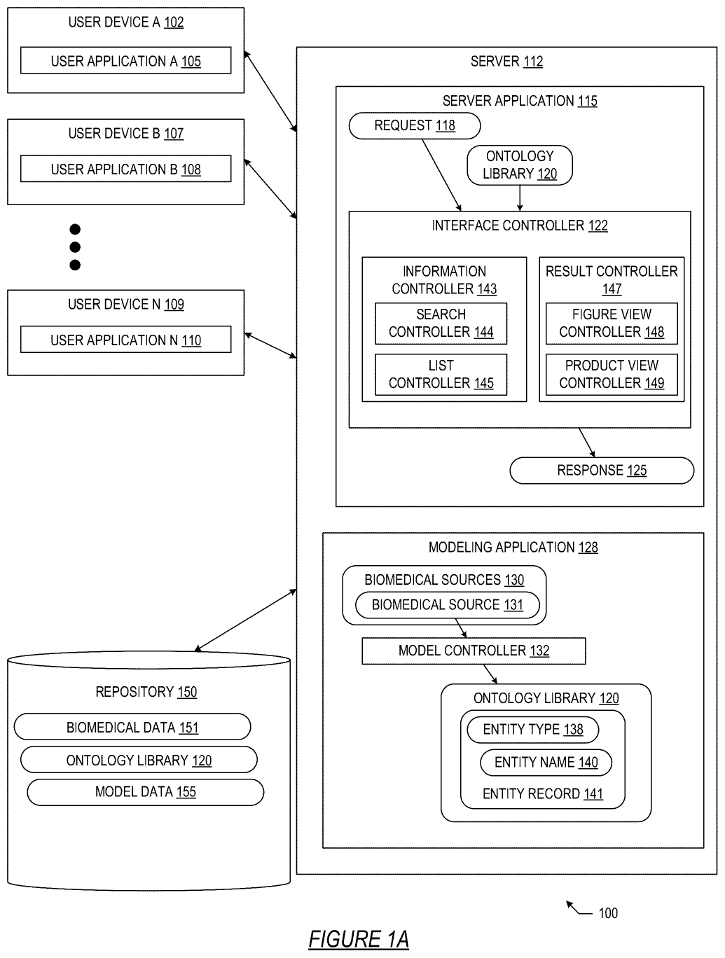

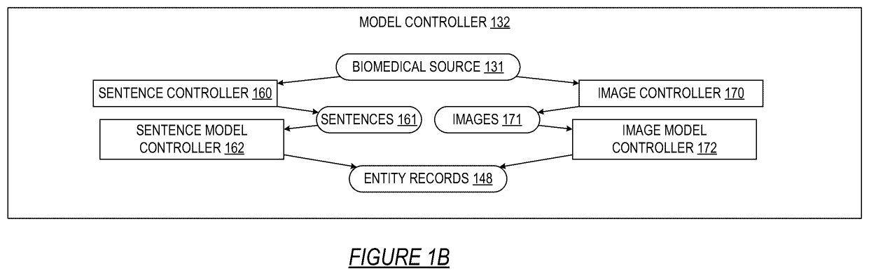

Turning to FIG. 1A, the system (100) selects reagents by interacting with users to search for and present biomedical information. The system (100) receives requests (e.g., the request (118)) and generates responses (e.g., the response (125)) using the ontology library (120). The system (100) generates the ontology library (120) from biomedical information (e.g., the biomedical sources (130)) stored in the biomedical data (151) using multiple machine learning and natural language processing models. The system (100) uses entity records of the ontology library (120) to populate search results with biomedical information from the biomedical sources (130) that are relevant to the requests from users. The system (100) generates the response (125) using the ontology library (120). The system (100) may display text, images, and information from the biomedical sources (130) to users operating the user devices A (102) and B (107) through N (109). The system (100) includes the user devices A (102) and B (107) through N (109), the server (112), and the repository (150).

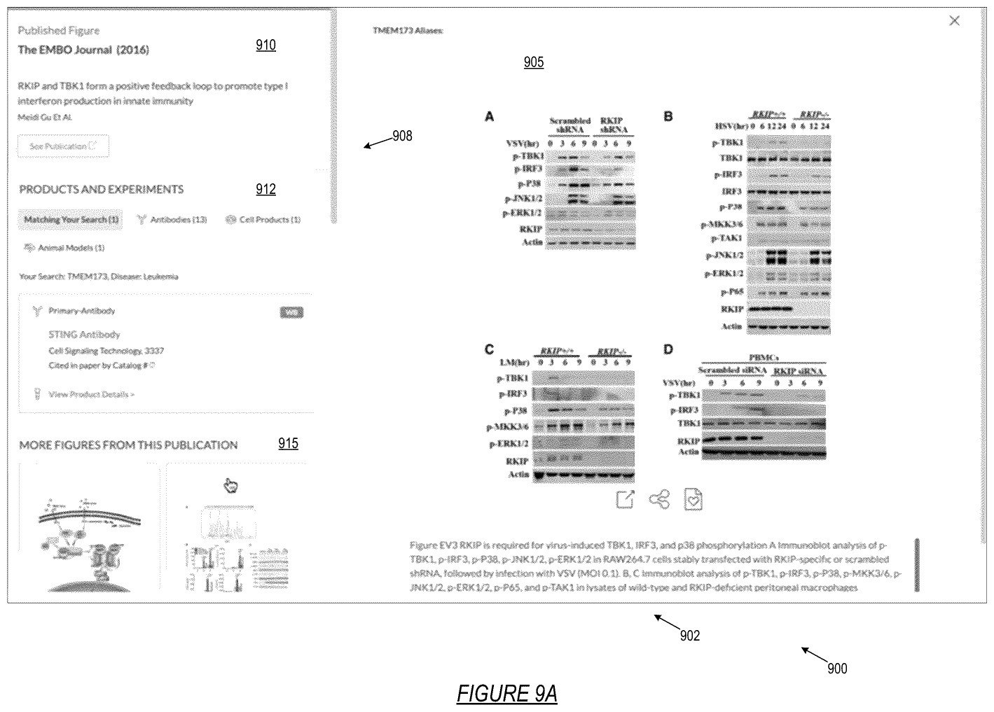

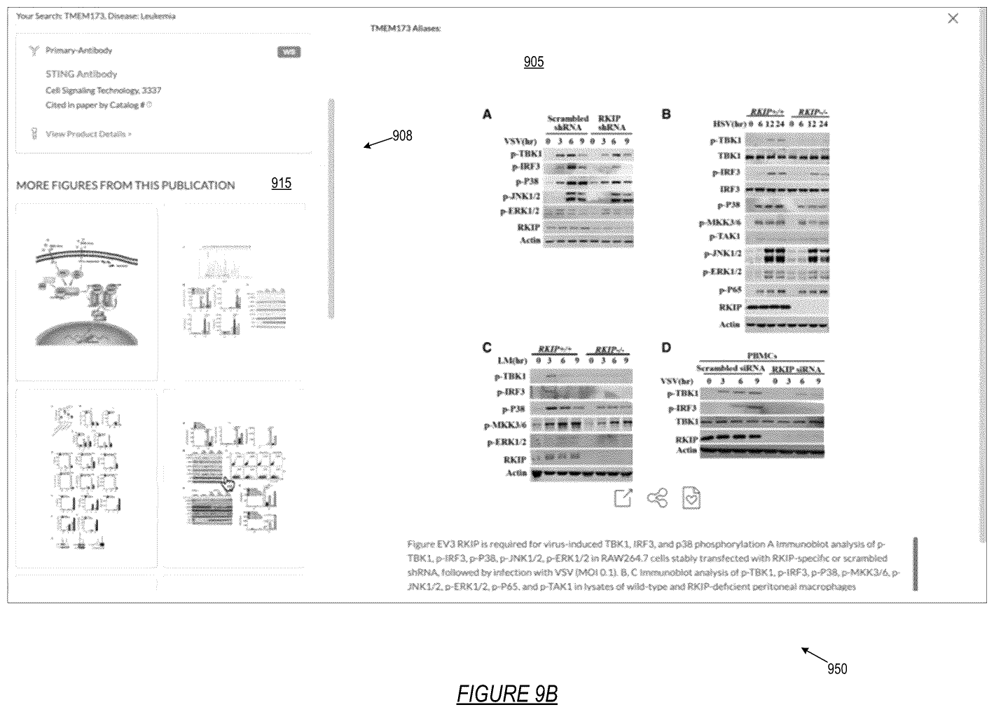

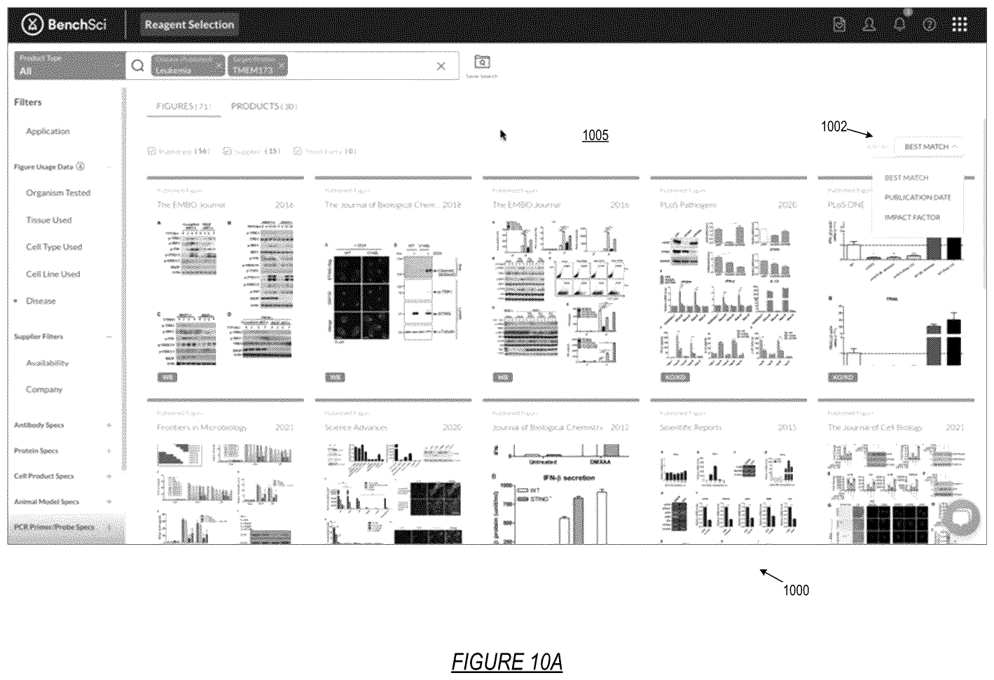

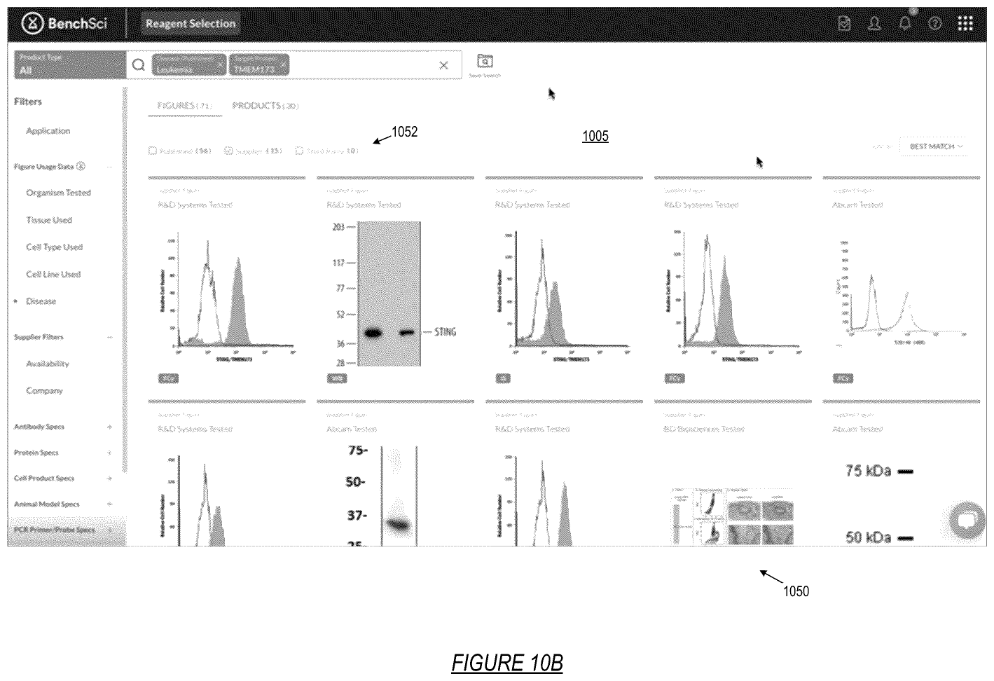

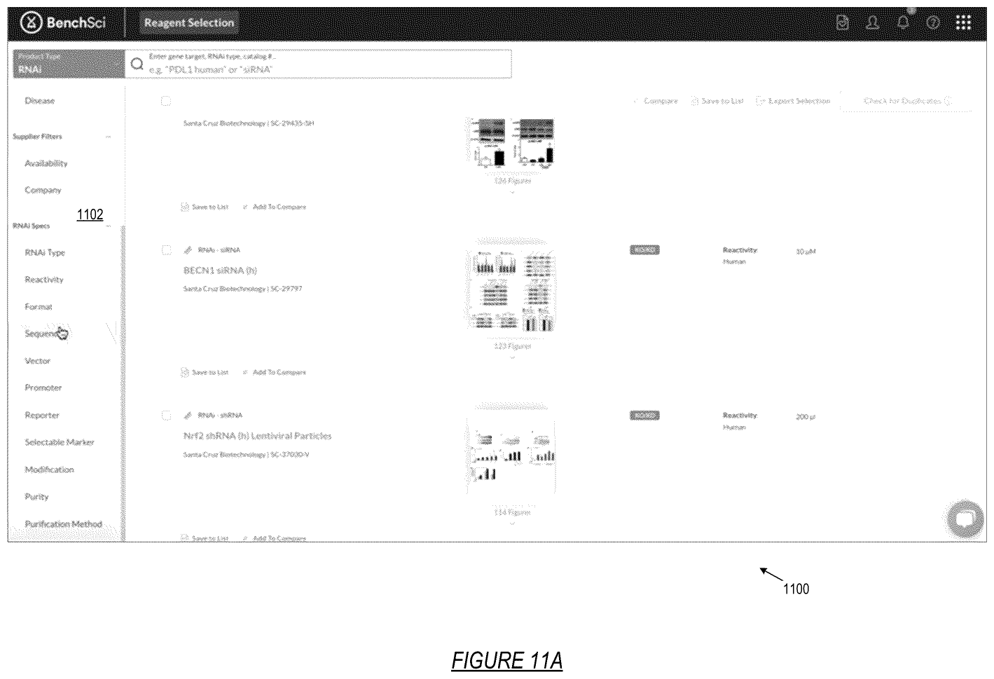

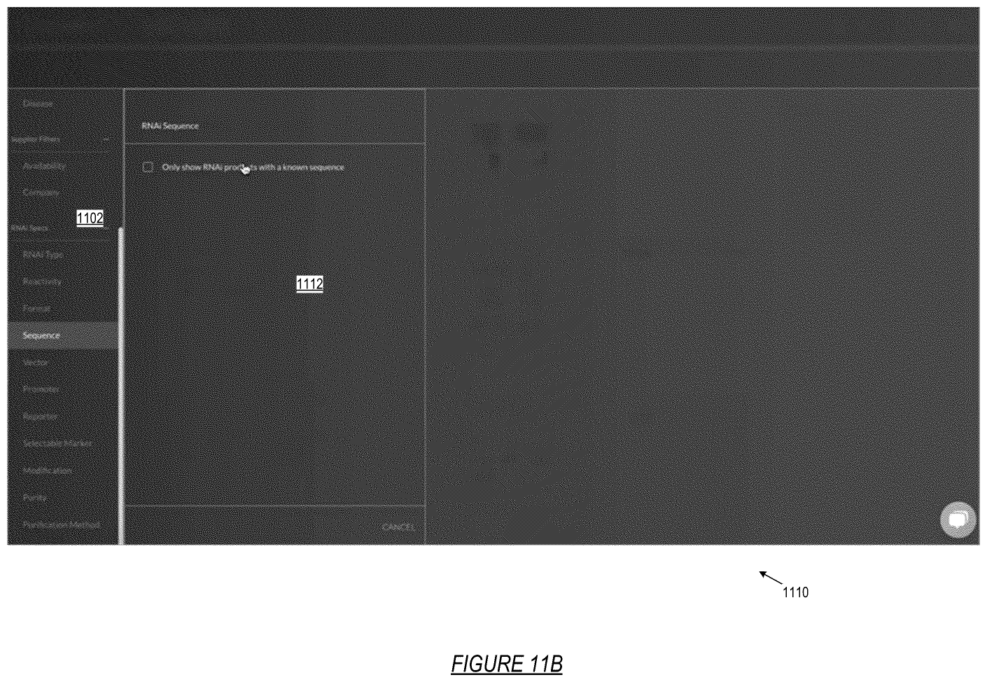

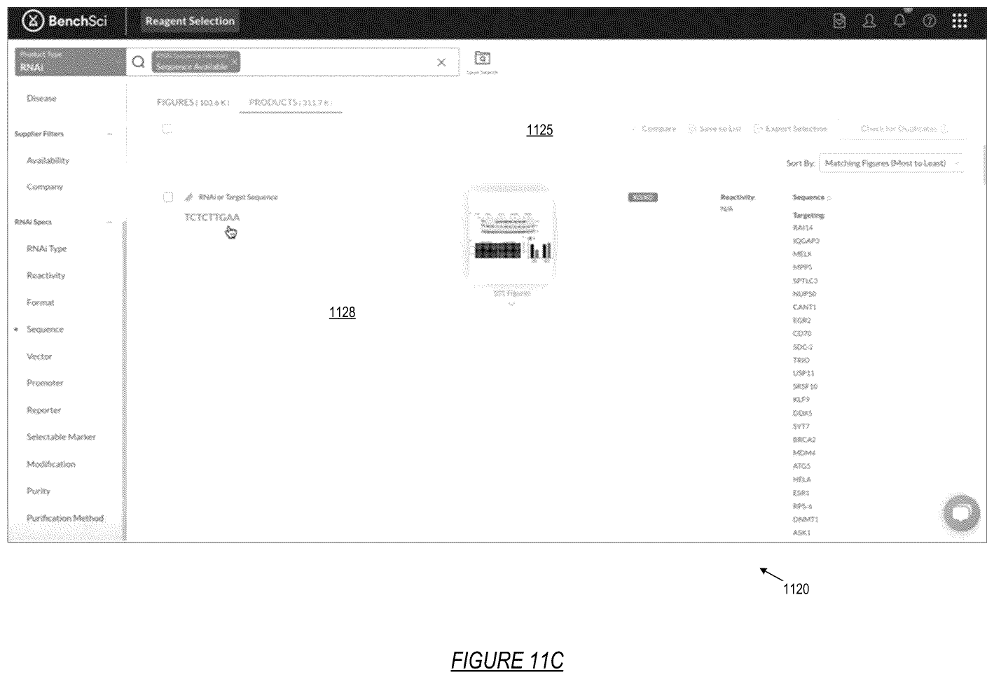

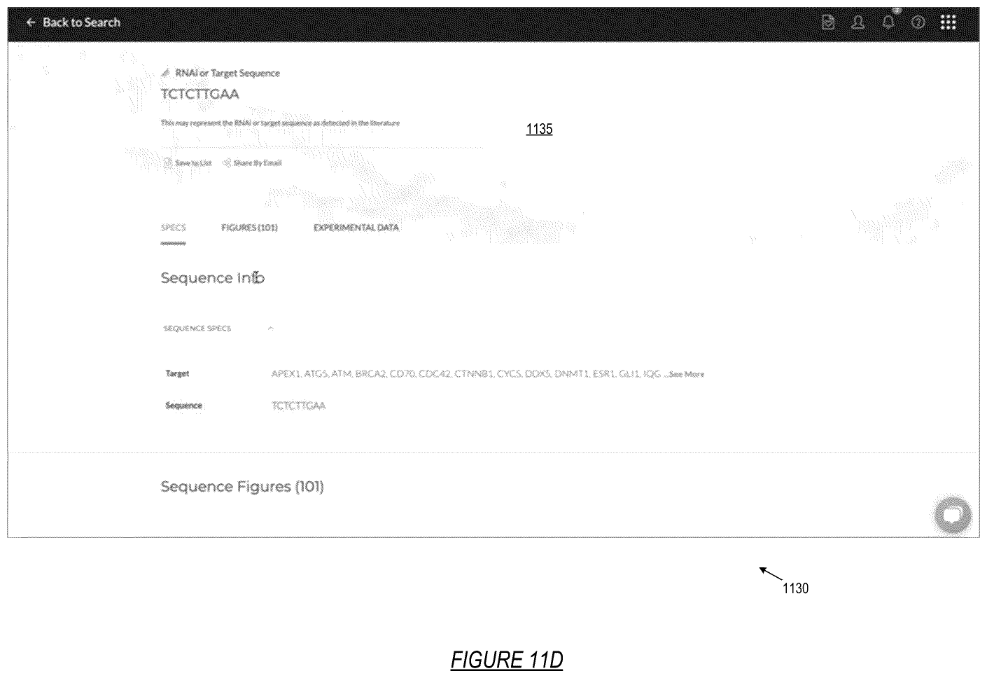

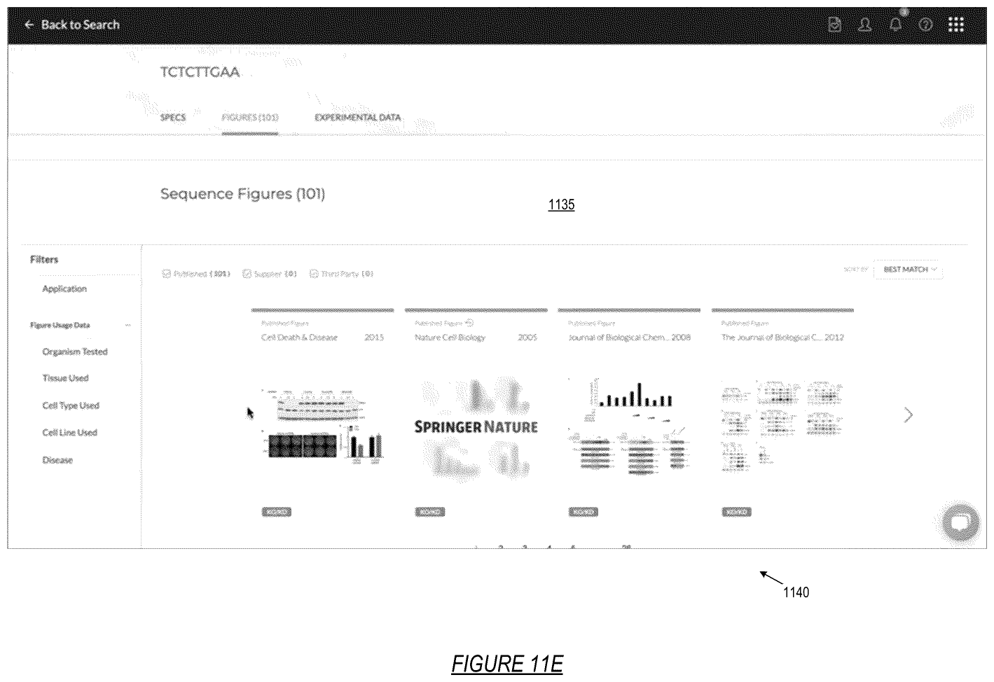

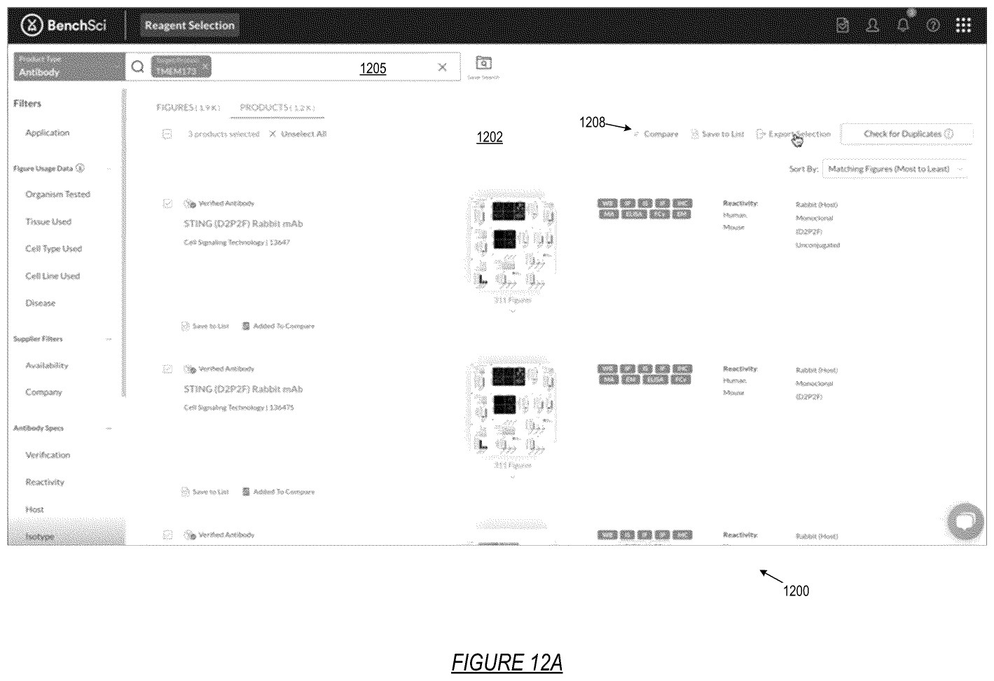

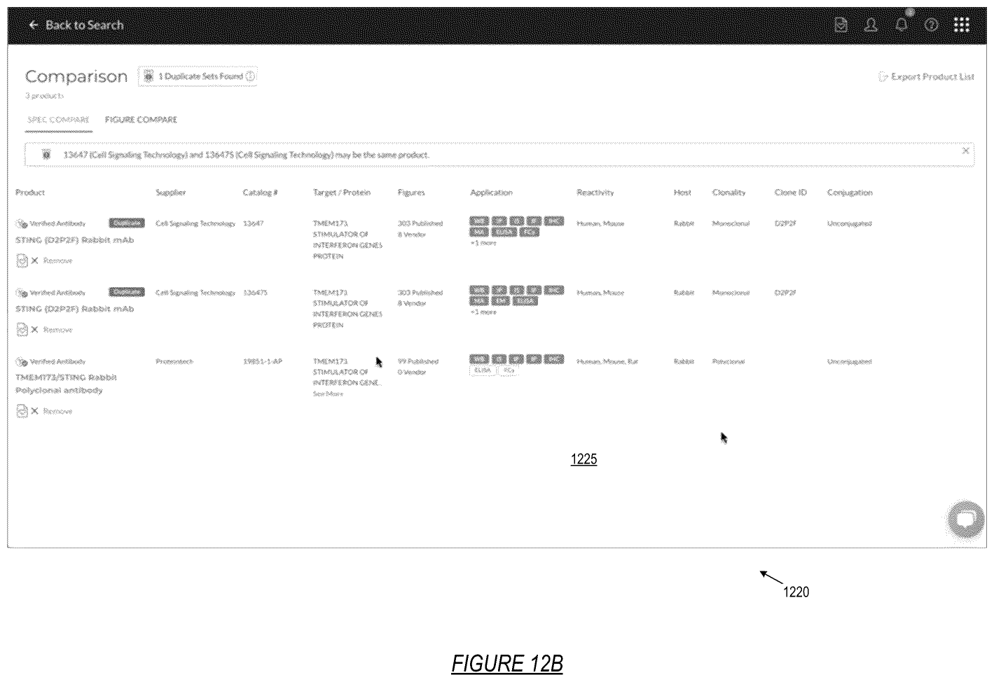

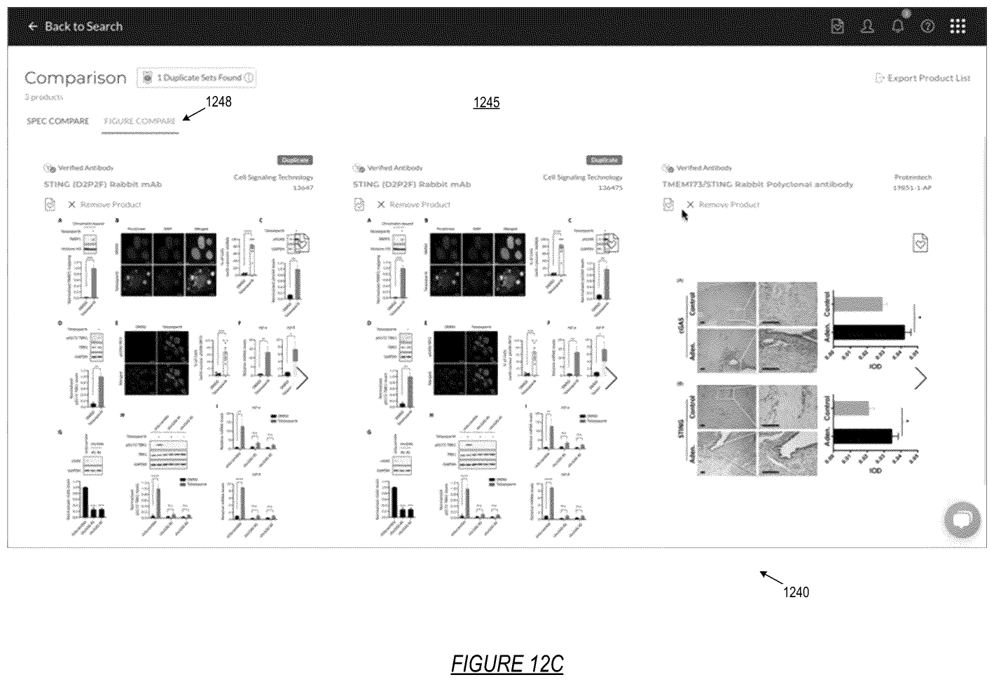

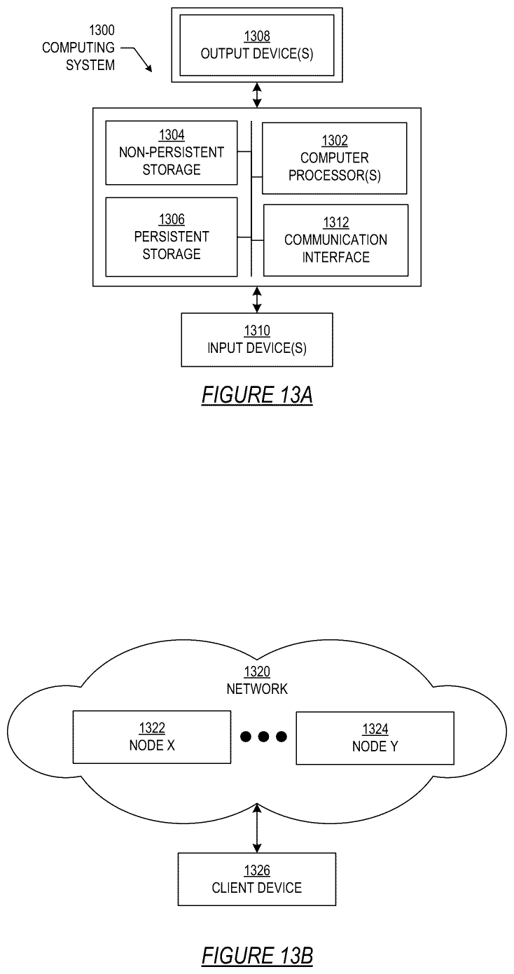

The server (112) is a computing system (further described in FIG. 13A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the ontology library (120) using the interface controller (122). The server application (115) may host websites accessed by users of the user devices A (102) and B (107) through N (109) to view and interact with information using the information controller (143) and the result controller (147). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the ontology library (120).

The request (118) is a request from one of the user devices A (102) and B (107) through N (109). In one embodiment, the request (118) is a search request to identify information (referred to as biomedical information) from the biomedical sources (130) that is relevant to one or multiple entities specified in the request (118). The biomedical information may include one or more entities defined in the ontology library (120), described in the biomedical data (151).

The ontology library (120) is generated with the modeling application (128), described further below. The ontology library (120) includes entity records (e.g., the entity record (141), described further below). The ontology library (120) may be stored in the repository (150).

The interface controller (122) is a collection of programs that may operate on the server (112). The interface controller (122) processes the request (118) using the ontology library (120) to generate the response (125). In one embodiment, the interface controller (122) searches the ontology library (120) to identify entity records and corresponding biomedical sources that include information that corresponds to the request (118). The interface controller (122) uses the information controller (143) and the result controller (147) to respond to the request (118).

The information controller (143) is a collection of programs that may operate on the server (112). The information controller (143) presents and stores biomedical information with interactive user interface elements to search the ontology library (120). The information controller (143) includes the search controller (144) and the list controller (145).

The search controller (144) is a collection of programs that may operate on the server (112). In one embodiment, the search controller (144) interacts with user interface elements presented on the user devices A (102) and B (107) through N (109). Interaction with the user interface elements of the search controller (144) specifies the entity types and names in the request (118).

The list controller (145) is a collection of programs that may operate on the server (112). In one embodiment, list controller (145) interacts with user interface elements presented on the user devices A (102) and B (107) through N (109). Interaction with the user interface elements of the list controller (145) presents categories and types of entities that may be used to search the biomedical data (151) for biomedical products relevant to the request (118).

The result controller (147) is a collection of programs that may operate on the server (112). The result controller (147) presents results from searches of the biomedical data (151) using the ontology library (120). The result controller (147) uses the figure view controller (148) to present figures from the biomedical data (151) relevant to the request (118) and uses the product view controller (149) to present information of products relevant to the request (118).

The figure view controller (148) is a collection of programs that may operate on the server (112). The figure view controller (148) presents figures from the biomedical data (151) that are relevant to the request (118).

The product view controller (149) is a collection of programs that may operate on the server (112). The product view controller (149) presents information from the biomedical data (151) that is relevant to the request (118).

The response (125) is generated by the interface controller (122) in response to the request (118) using the ontology library (120). In one embodiment, the response (125) includes images from the biomedical data (151). Portions of the response (125) may be displayed by the user devices A (102) and B (107) through N (109) that receive the response (125).

The modeling application (128) is a collection of programs that may operate on the server (112). The modeling application (128) generates the ontology library (120) from the biomedical sources (130).

The biomedical sources (130), including the biomedical source (131), are collections of biomedical information from the biomedical data (151). The biomedical source (131) may be a publication of biomedical research that describes and provides evidence of biomedical interactions between entities. For example, a biomedical source may describe an experiment testing the efficacy of an antibody (a biomedical product) in combatting a disease. Both the antibody and the disease are entities defined in the ontology library (120). The biomedical source (131) may include text and images that are processed by the model controller (132). As another example, entities that are proteins may suppress or enhance the expression of other entities and affect the prevalence of certain diseases.

The model controller (132) is a collection of programs that may operate on the server (112). The model controller (132) processes the biomedical sources (130) to generate the ontology library (120). The model controller (132) uses models, including machine learning and natural language processing models, to identify entities within the biomedical sources (130). The model controller is further described with FIG. 1B.

The ontology library (120) is a collection of entity records, including the entity record (141). The ontology library (120) may be stored in the repository (150).

The entity record (141) is a record of an entity. The entity record (141) defines the types and names of entities that are recognized by the system (100).

The entity type (138) identifies the type of the entity represented by the entity record (141). The entity type (138) may be stored using strings, categorical information, numerical information, etc. Types of entities include proteins, genes, diseases, experiment techniques, chemicals, cell lines, pathways, tissues, cell types, organisms, etc.

The entity name (140) identifies the name of the entity represented by the entity record (141). The entity name (140) may include a list of multiple aliases that are used to reference the same entity from the different biomedical sources (130).

The user devices A (102) and B (107) through N (109) are computing systems (further described in FIG. 13A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information, including information from the biomedical data (151). In one embodiment, the user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to search for information on biomedical products in the biomedical data (151). The user application A (105) may transmit search terms that identify one or multiple entities defined in the ontology library (120). The user application A (105) may receive the response (125) in response to the request (118) and display biomedical information from the response (125).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system (1300) and the nodes (1322) and (1324) described below in FIGS. 13A and 13B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the biomedical data (151), the ontology library (120), and the model data (155).

The biomedical data (151) includes biomedical information of which the biomedical sources (130) are a subset. The biomedical data (151) may include several electronic files stored in multiple databases. For example, the biomedical data (151) may include electronic copies of multiple research journals from different databases. The files of the biomedical data (151) may include image data and text data. The image data includes images that represent the graphical figures from the files. The text data represents the writings of biomedical information in the biomedical data (151). The text data for a file includes multiple sentences that each may include multiple words that each may include multiple characters stored as strings in the repository (150). In one embodiment, the biomedical data (151) includes biomedical information stored as extensible markup language (XML) files, portable document files (PDFs), etc. In one embodiment, the file formats define containers for the text and images of the biomedical information describing evidence of biomedical experiments.

The ontology library (120) includes information that of the entities and biomedical terms and phrases used by the system (100), which may be stored in entity records, including the entity record (141). Multiple terms and phrases may be used for the same entity. The ontology library (120) defines types of entities. In one embodiment, the types include the types of protein/gene, chemical, cell line, pathway, tissue, cell type, disease, organism, etc. The ontology library (120) may store the information about the entities in a database, structured text files, combinations thereof, etc.

The model data (155) includes the data for the models used by the system (100). The models may include rules-based models and machine learning models. The machine learning models may be updated by training, which may be supervised training. The modeling application (128) may load the models from the model data (155) to generate the entity record (141) of the ontology library (120) from the biomedical source (131).

The model data (155) may also include intermediate data. The intermediate data is data generated by the models during the process of generating the entity record (141) from the biomedical source (131).

Although shown using distributed computing architectures and systems, other architectures and systems may be used. In one embodiment, the server application (115) may be part of a monolithic application that manipulates biomedical information. In one embodiment, the user applications A (105) and B (108) through N (110) may be part of monolithic applications that manipulate biomedical information without the server application (115).

Turning to FIG. 1B, the model controller (132) generates the entity records (148) from the biomedical source (131). A single biomedical source may include references to multiple entities using both text, figures, text within the images, etc. The model controller (132) includes the sentence controller (160), the image controller (170), the sentence model controller (162), and the image model controller (172).

The sentence controller (160) is a collection of programs that may operate on the server (112). The sentence controller (160) processes the biomedical source (131) to extract the sentences (161).

The sentences (161) are sentences of text from the biomedical source (131). The sentences (161) may be preprocessed and formatted for input to the machine learning models of the sentence model controller (162).

The image controller (170) is a collection of programs that may operate on the server (112). The image controller (170) processes the biomedical source (131) to extract the images (171).

The images (171) are images of figures from the biomedical source (131). The images (171) may be preprocessed and formatted for input to the machine learning models of the image model controller (172).

The sentence model controller (162) processes the sentences (161) to generate a portion of the entity records (148) using one or multiple machine learning models. The machine learning models may include neural networks using long short-term memory (LSTM), transformers, attention, etc. The sentence model controller (162) may train the machine learning models by comparing the output from the models to labels that identify the expected output for a given input and using backpropagation, gradient descent, etc., to update the machine learning models based on the comparison.

The image model controller (172) processes the images (171) to generate a portion of the entity records (148) using one or multiple machine learning models. The machine learning models may include neural networks using convolutional networks, transformers, attention, etc. The image model controller (172) may train the machine learning models by comparing the output from the models to labels that identify the expected output for a given input and using backpropagation, gradient descent, etc., to update the machine learning models based on the comparison.

The entity records (148) are generated from the sentences (161) and the images (171). The entity records (148) form a portion of the ontology library (120) of FIG. 1A.

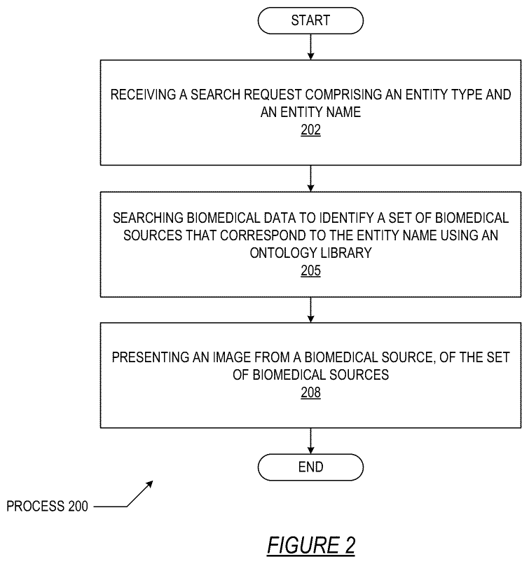

Turning to FIG. 2, the process (200) is for reagent selection. The process (200) may be performed by a computing system, such as the computing system (1300) of FIG. 13A.

At Step 202, a search request is received that includes an entity type and an entity name. In one embodiment, the search request is received by a user interface in response to interaction from a user and then transmitted to a server that receives the request. The request may specify multiple entities to use for searching for biomedical information using an ontology library. The entities are identified by the types and names specified in the request.

At Step 205, biomedical data is searched to identify a set of biomedical information sources that correspond to the entity name using an ontology library. In one embodiment, a text search may be used to identify entity names (or alias thereof) that occur in the biomedical sources from the biomedical data. The system may track which biomedical sources include the names (or aliases) and then present information from the tracked biomedical sources to the user device that sent the search request.

In one embodiment, multiple biomedical sources of the biomedical data are ingested to generate the ontology library. In one embodiment, the set of biomedical sources is a subset of the biomedical data. In one embodiment, the ontology library defines multiple entitles using entity types and entity names. The entity type and name from the search request are defined in the ontology library and then used to search the biomedical data.

In one embodiment, prior to receiving the search request, the biomedical source is processed to record a correspondence between the entity type, the entity name, and the biomedical source. In one embodiment, the entity records of the ontology library may record the biomedical sources that include the name (or aliases) of the entity.

In one embodiment, {205; claim 6} prior to receiving the search request, at least a portion of the image is processed with a machine learning model to identify a type of an entity, defined in the ontology library, corresponding to at least a portion of the image. One or multiple machine learning models may be used to classify the type of entity from the portion of the image. The machine learning models may include convolutional neural networks. In one embodiment, the machine learning models identify text from the image and the text may be processed to identify the names and aliases of entities defined by the ontology library.

In one embodiment, prior to receiving the search request, text from the biomedical source is processed with a machine learning model to identify a type of an entity, defined in the ontology library, corresponding to the text. One or multiple machine learning models may be used to classify words and phrases from the text as the entities defined in the ontology library. The machine learning models may include recurrent neural networks, long short-term memories (LS TMs), transformer networks, attention networks, combinations thereof, etc.

At Step 208, an image from a biomedical information source is presented that corresponds to the entity name from the search request. In one embodiment, the image is presented by transmitting the image from a server to a user device, which displays the image.

In one embodiment, the image is presented in a figure view. In one embodiment, the figure view includes a set of images that correspond to the set of biomedical sources identified using the ontology library.

In one embodiment, the image is presented in a product view. In one embodiment, the product view includes a set of rows with a row that includes the image and product information in a plurality of fields.

In one embodiment, images corresponding to multiple biomedical sources are presented. The ordering of the images may use one of multiple of criteria, including a match criterion, a date criterion, an impact criterion, etc.

The match criterion orders the images using a number of matches of the entity name to text from the biomedical source. A biomedical source that includes multiple uses of the entity name may be ranked higher with the match criterion than a biomedical source that has fewer uses of the entity name.

The date criterion orders the images using a date of the biomedical source. A biomedical source with a newer or more recent date may be ranked higher with the date criterion than a biomedical source with an older date.

The impact criterion orders the images using a number of citations to the biomedical source. A biomedical source with a more citations by other biomedical sources may be ranked higher with the impact criterion than a biomedical source with fewer citations by other biomedical sources. For example, a biomedical source that has been cited to by five other biomedical sources may be ranked higher than a biomedical source that has been cited to by one other biomedical source.

In one embodiment, the search request is received in response to selection of the entity name presented in a category presented in an entity type presented in a filter view. The filter view may be transmitted to and displayed by a user device. The user may interact with the filter view to select, using the filter view, an entity type, a category, and an entity name. In one embodiment, the selections are received by the user device and transmitted to a server. The server may update a set of search results using the selection.

In one embodiment, a specification view of a product specification is presented in response to selection of a product identifier. The specification view presents detail information about a biomedical product. For example, the detail information may include the vendor name and vendor SKU for the product. Presentation of the specification view may be triggered from a source view or a product view.

In one embodiment, a source view of a biomedical source is presented. The source view may be presented after clicking on a figure from a figure view or from clicking on a row of a product view.

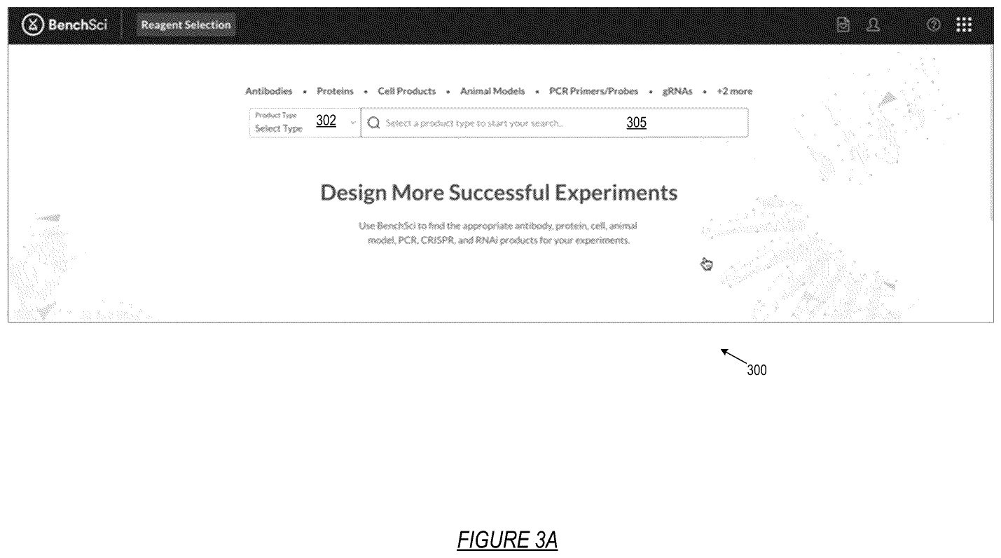

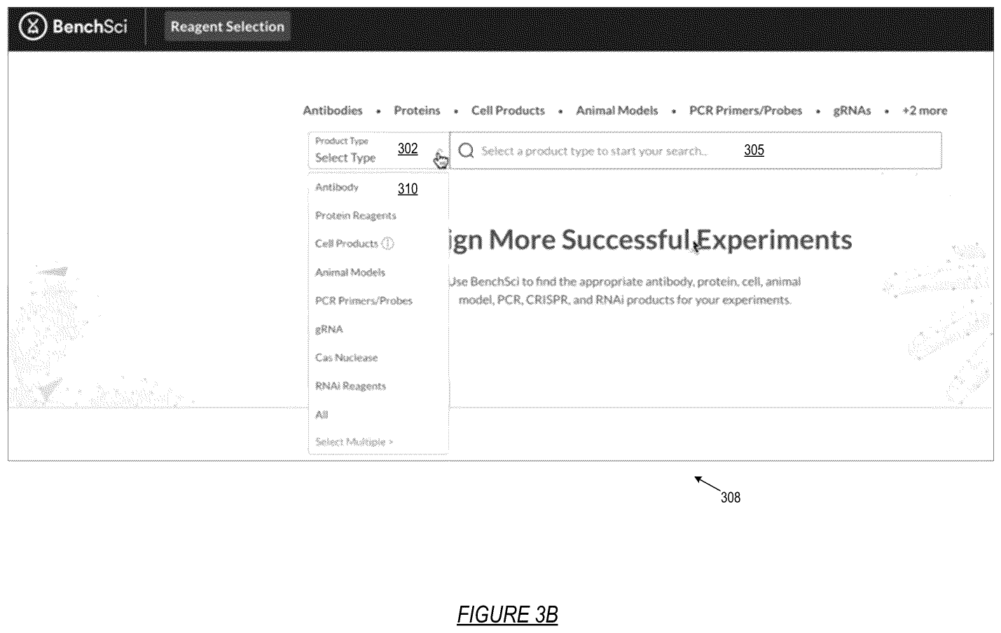

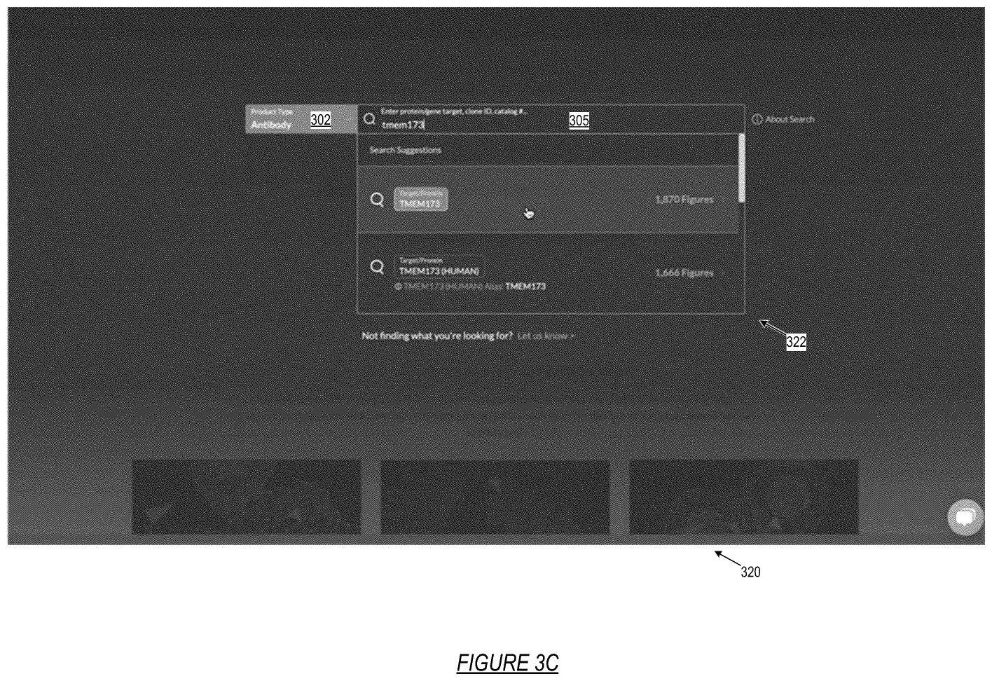

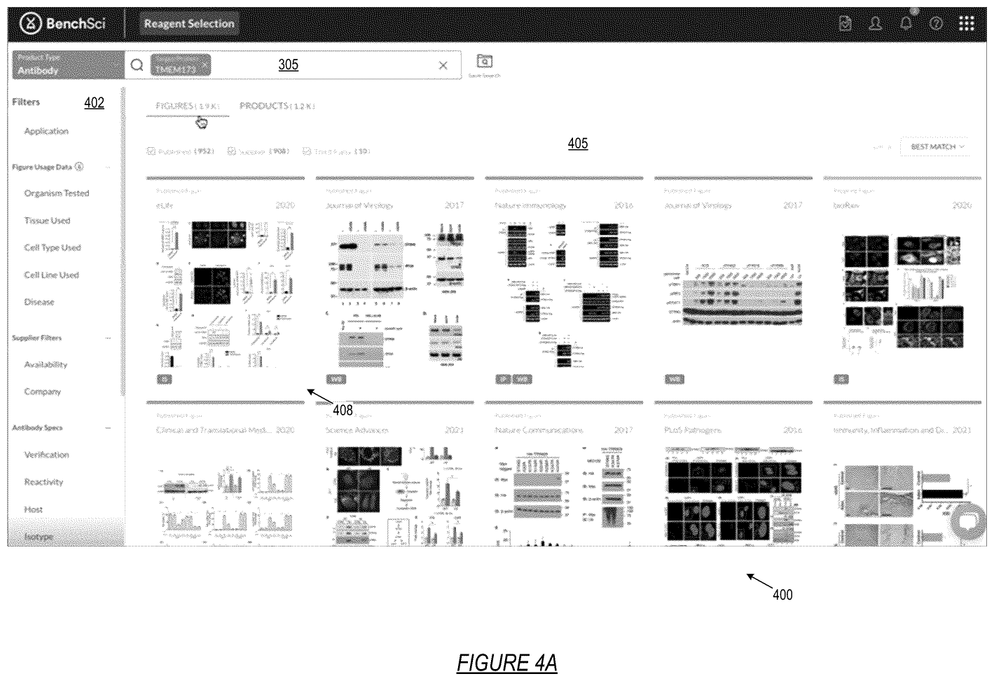

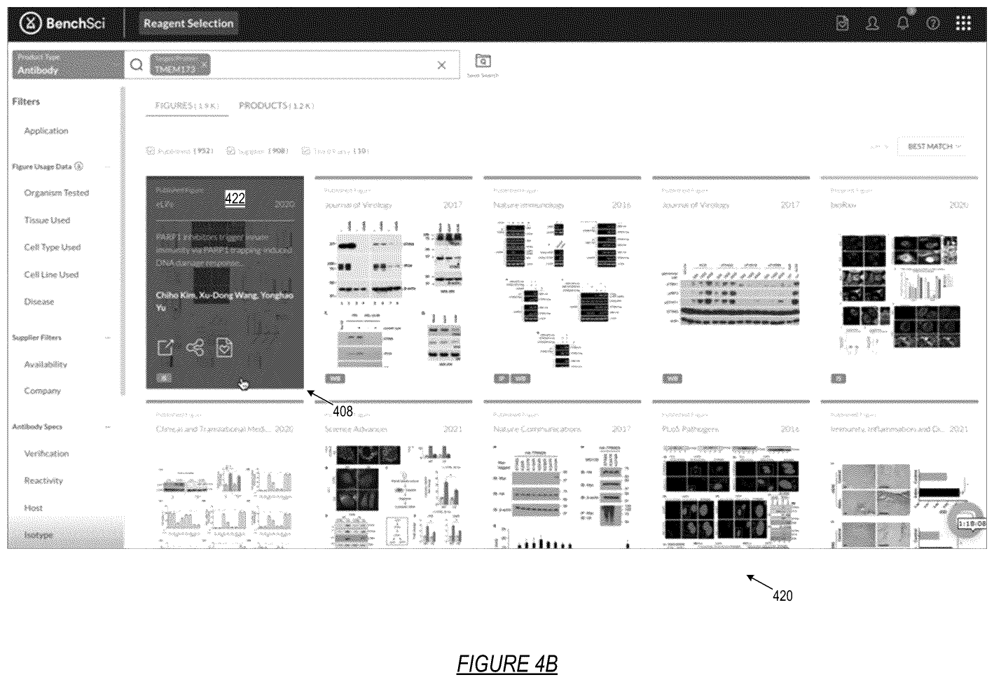

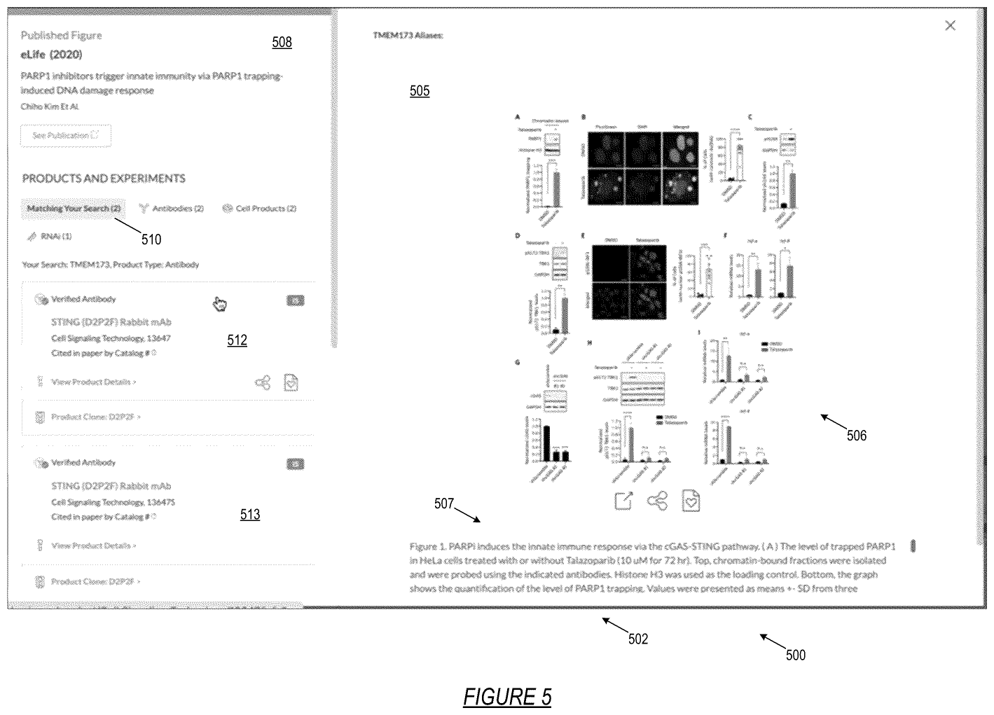

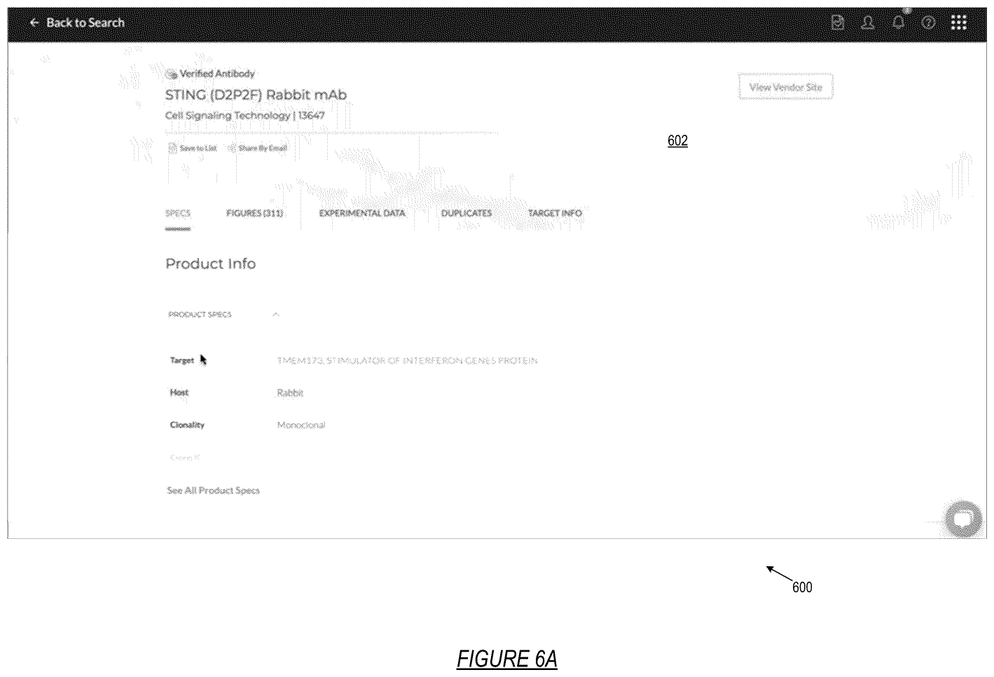

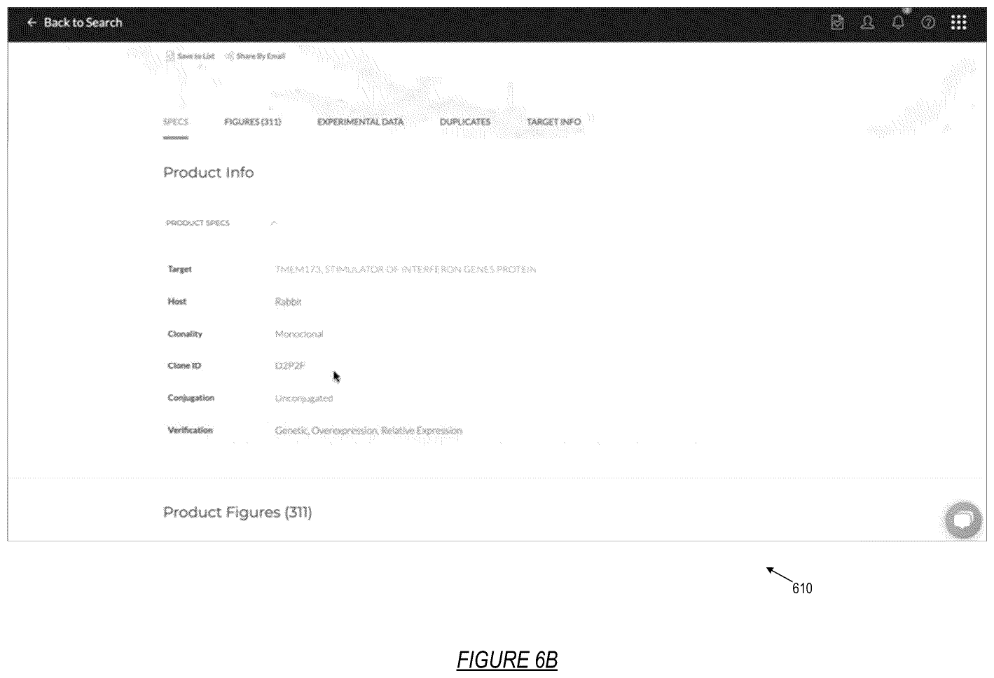

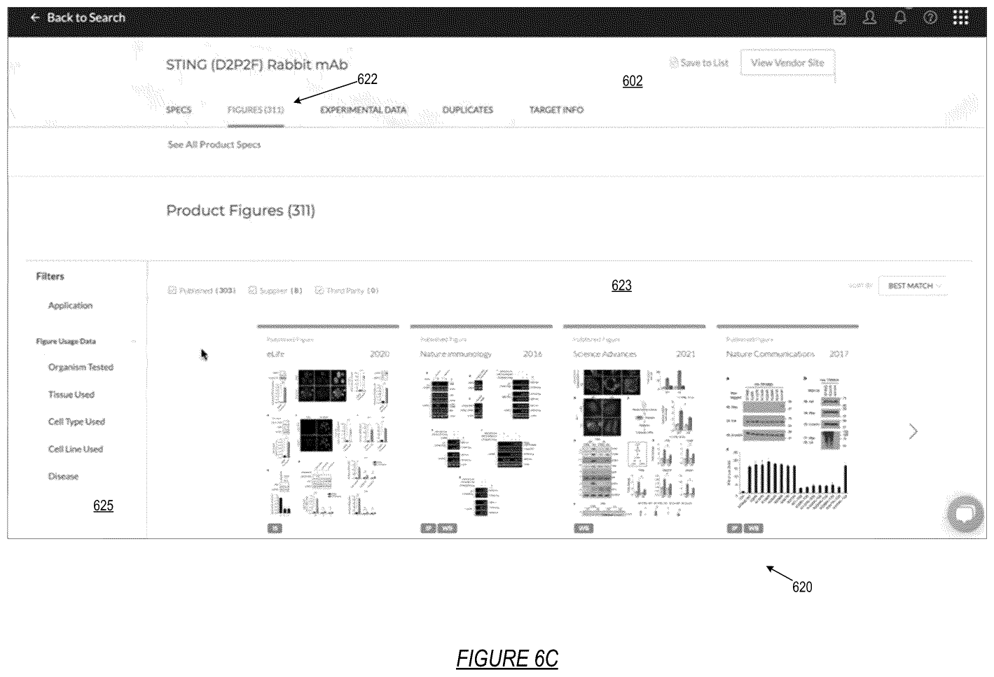

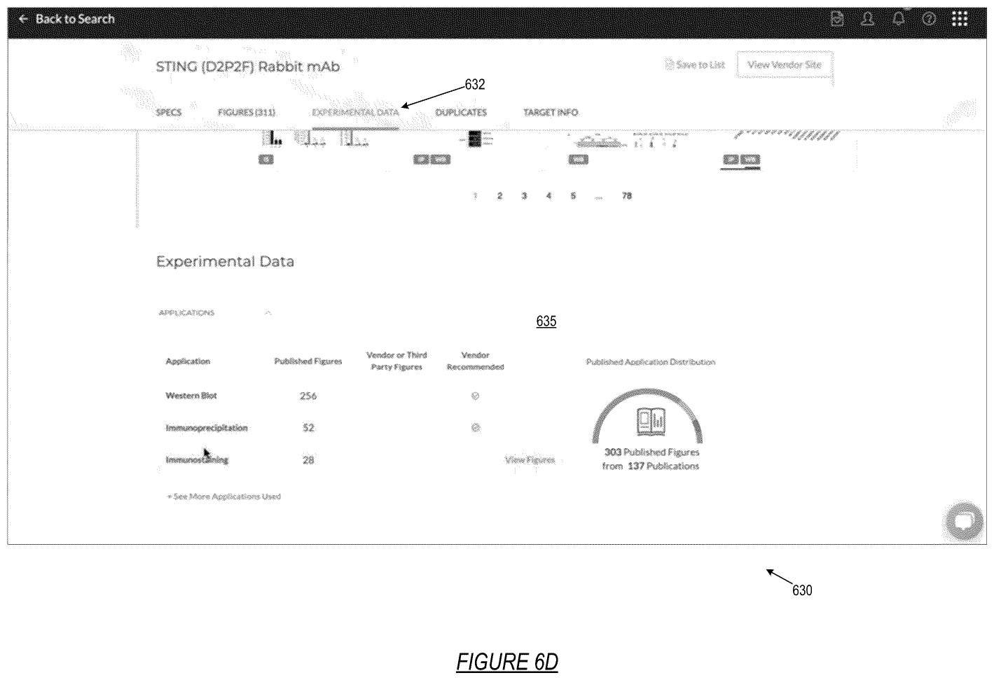

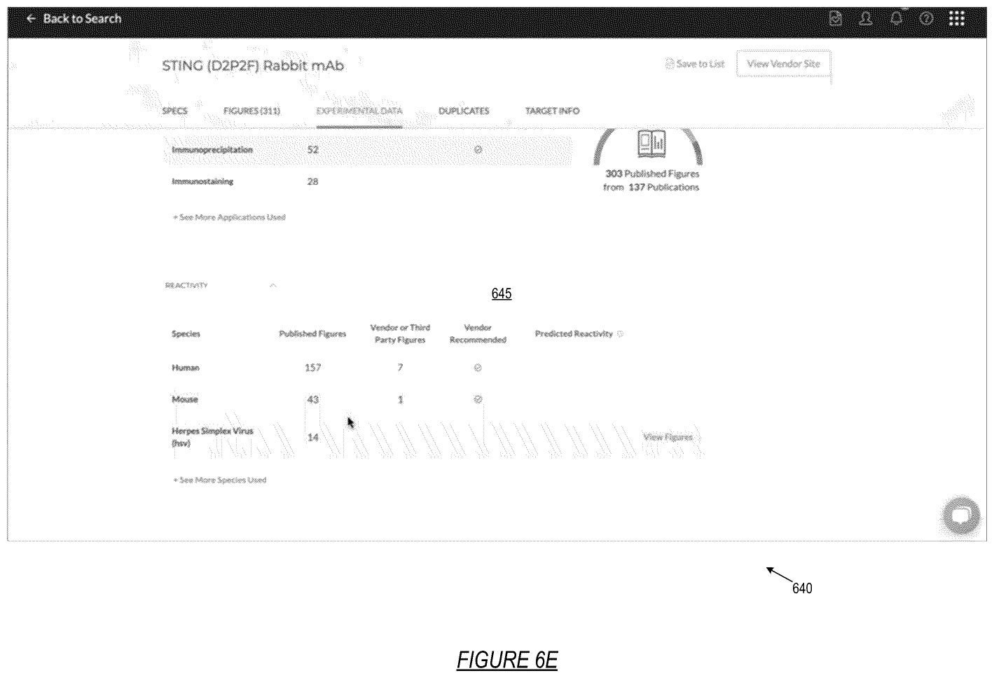

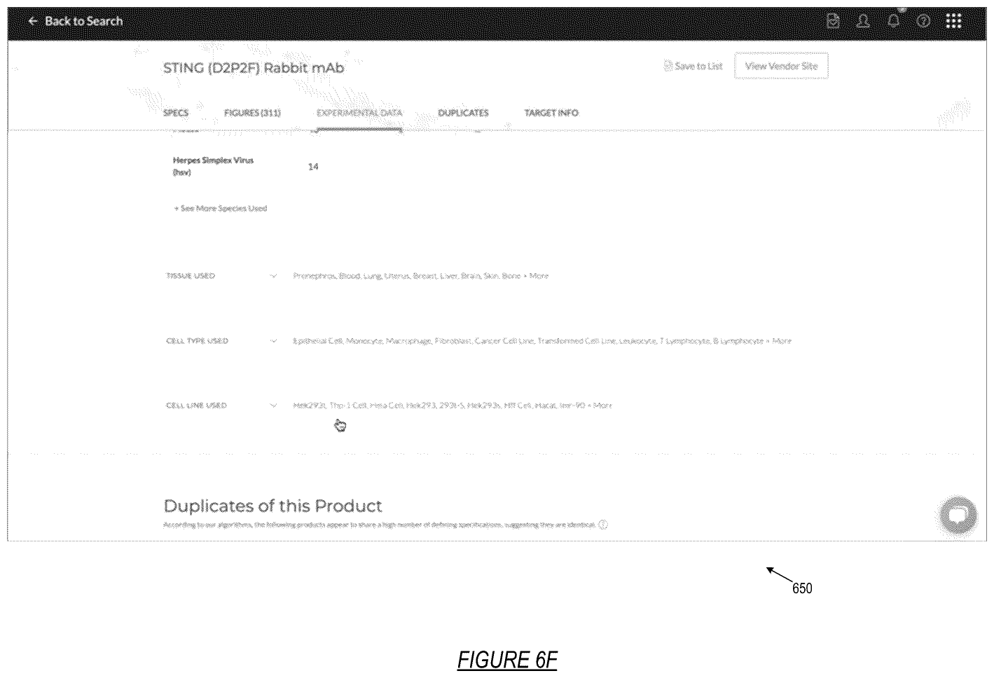

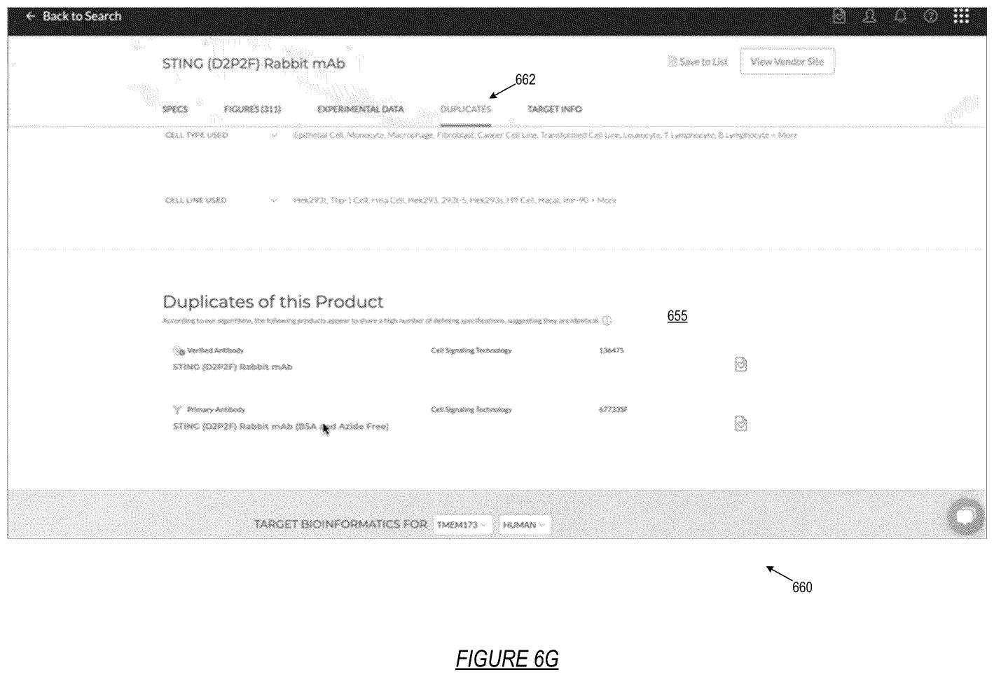

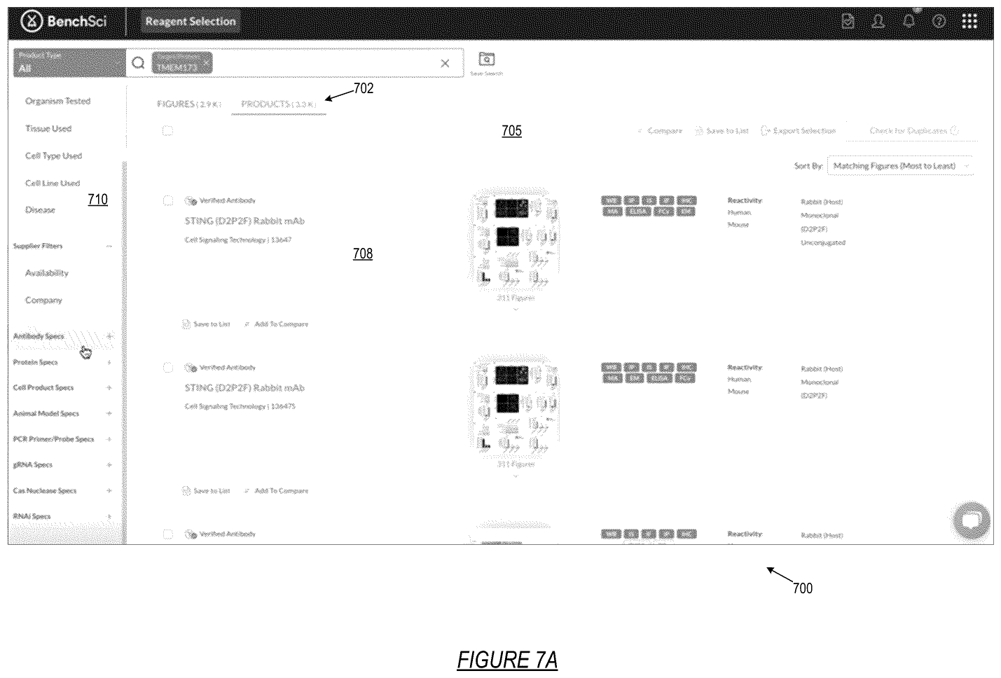

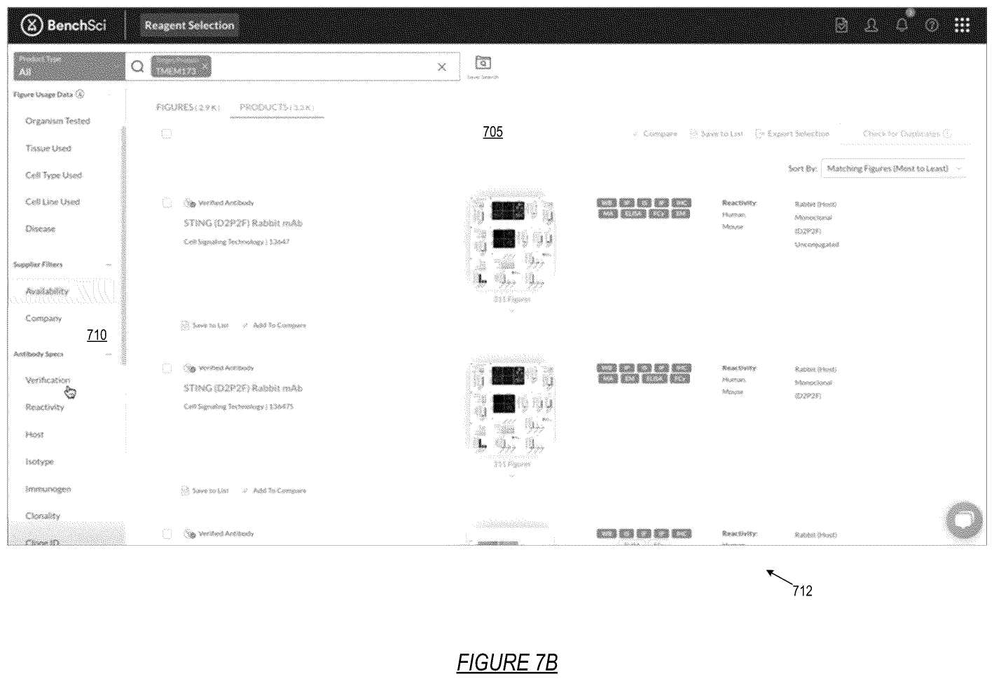

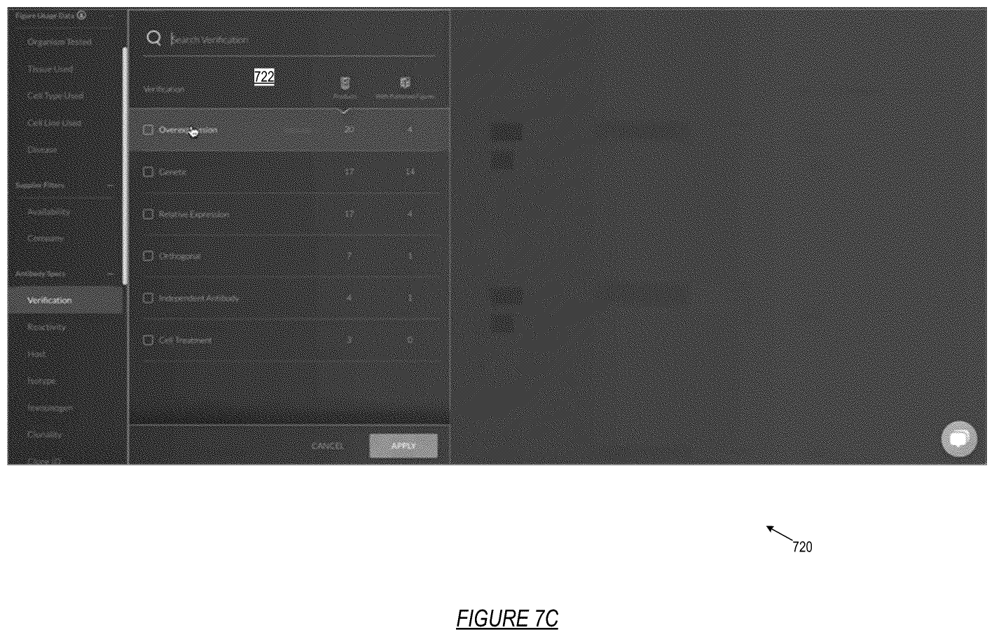

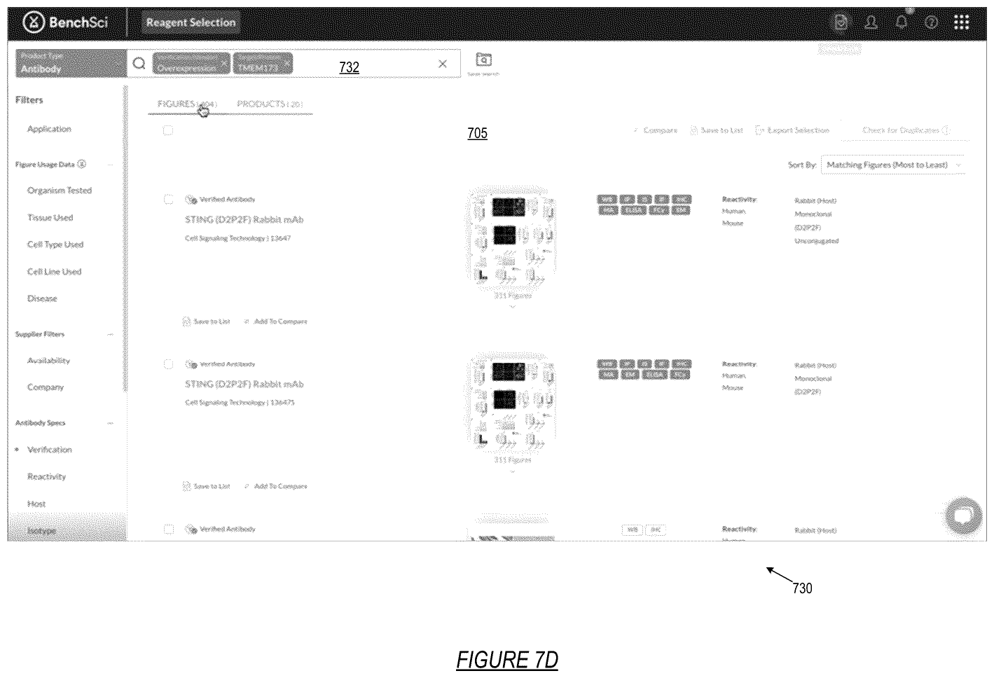

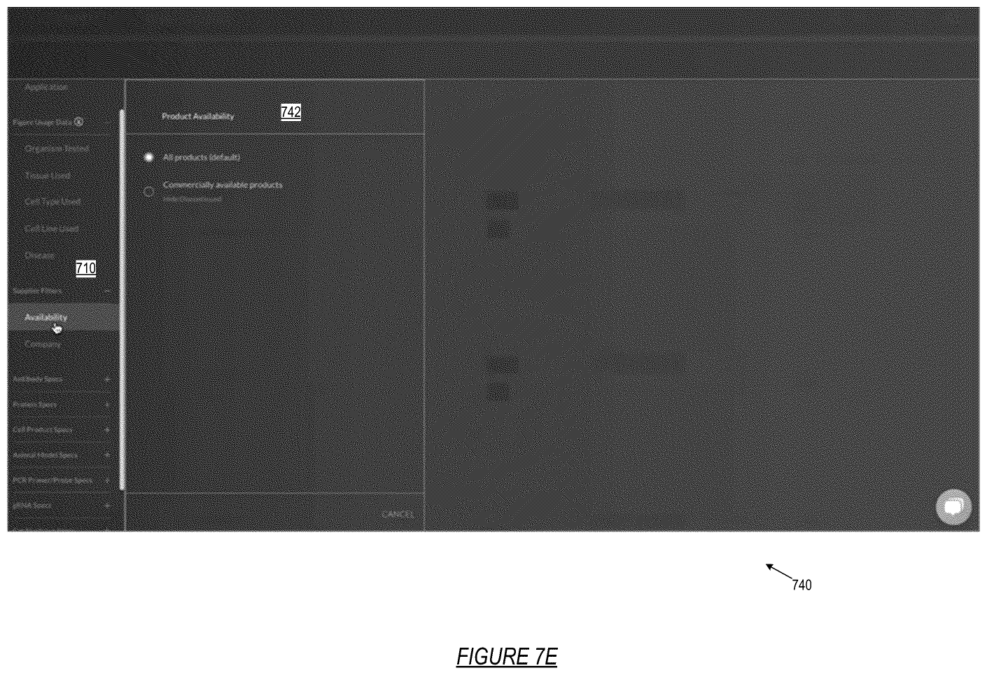

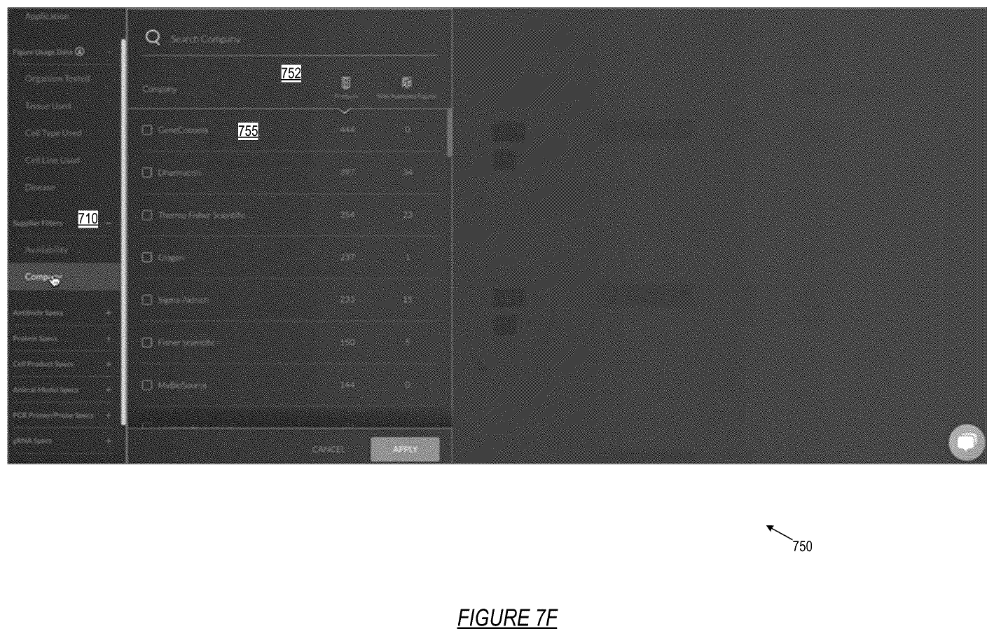

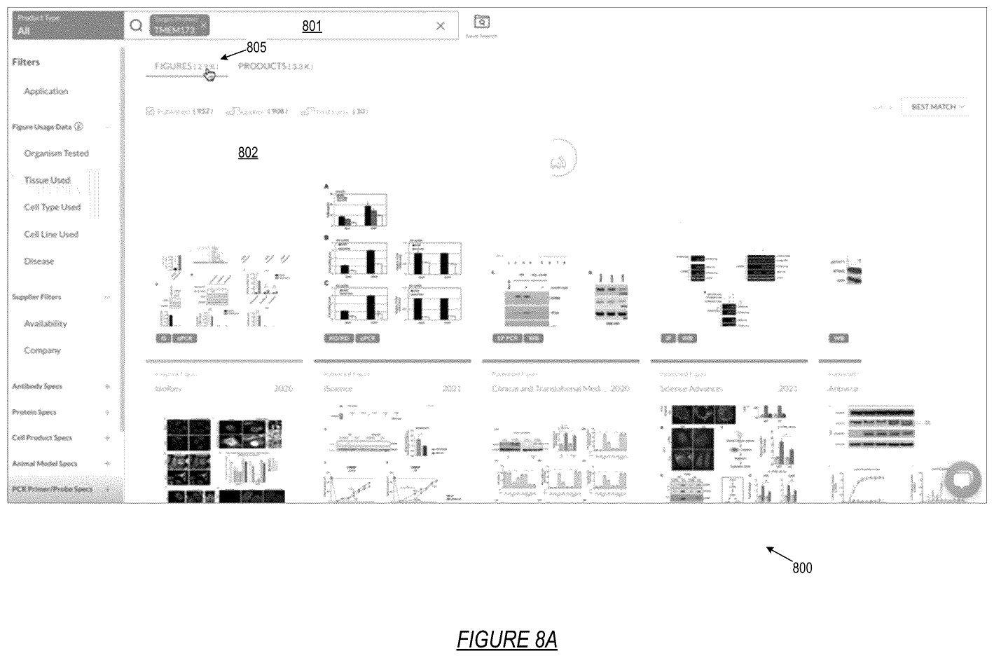

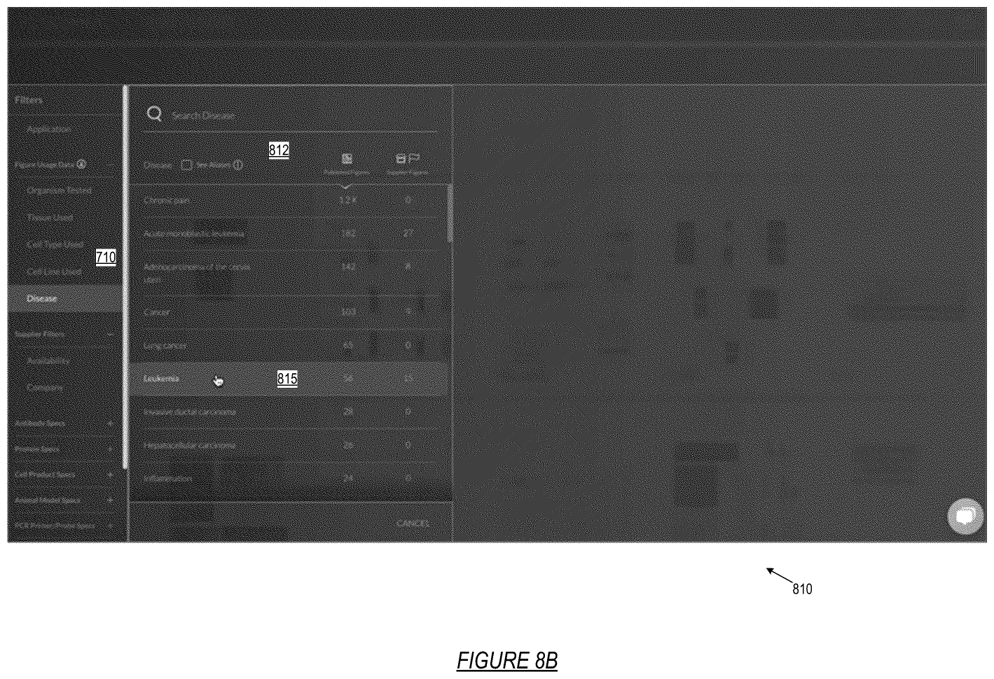

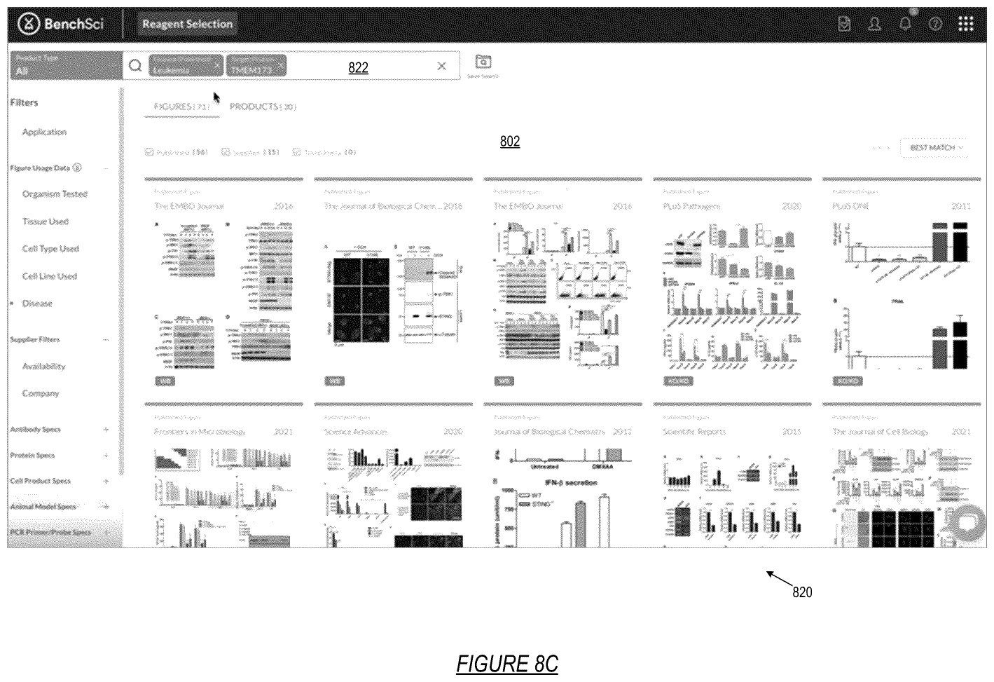

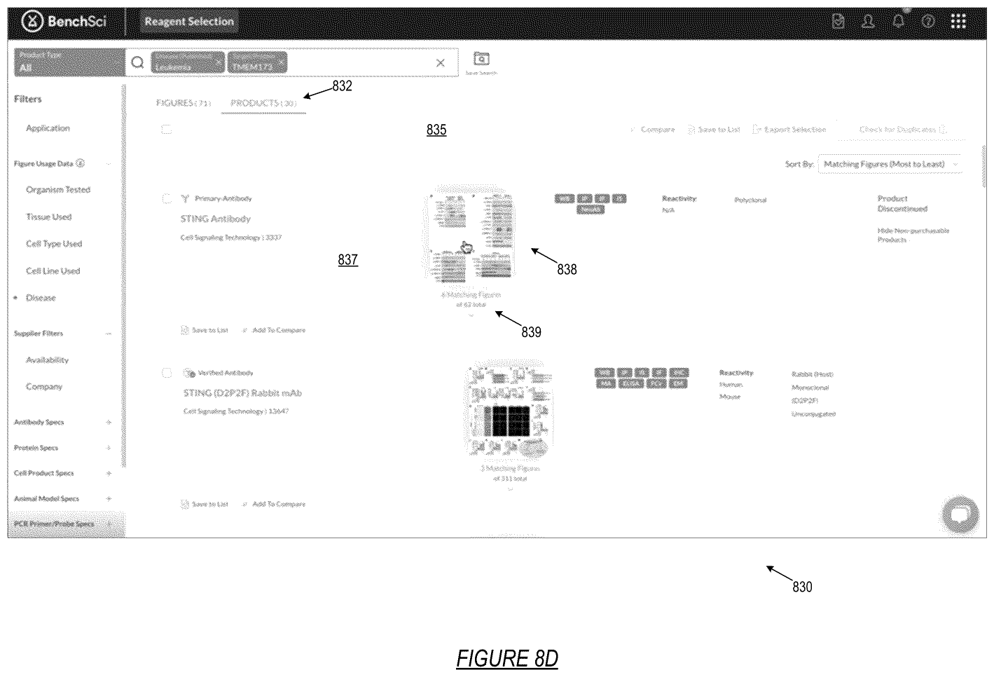

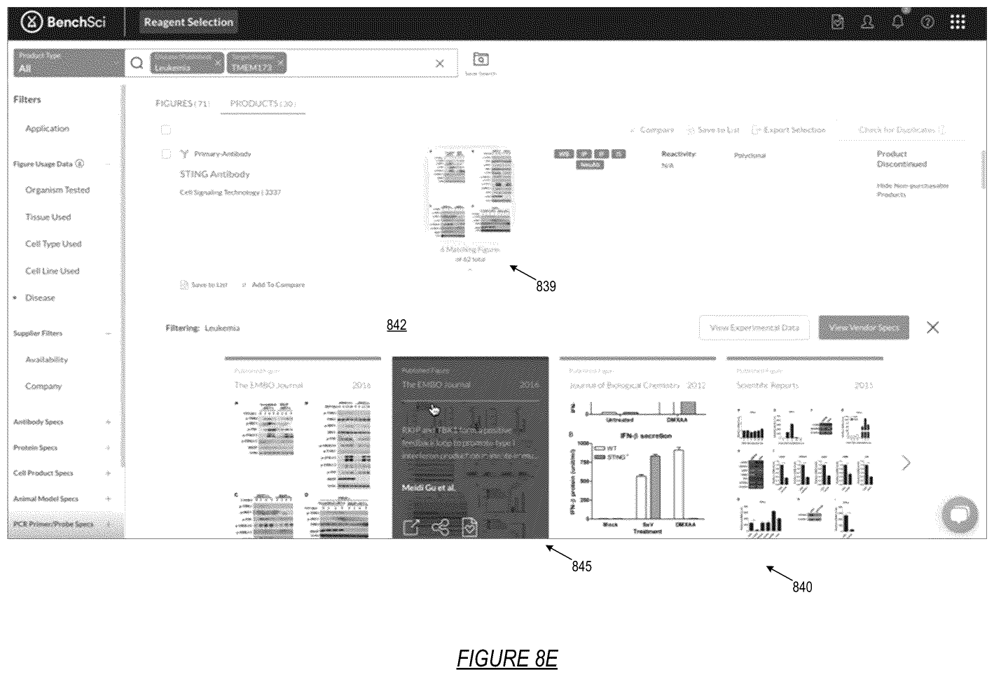

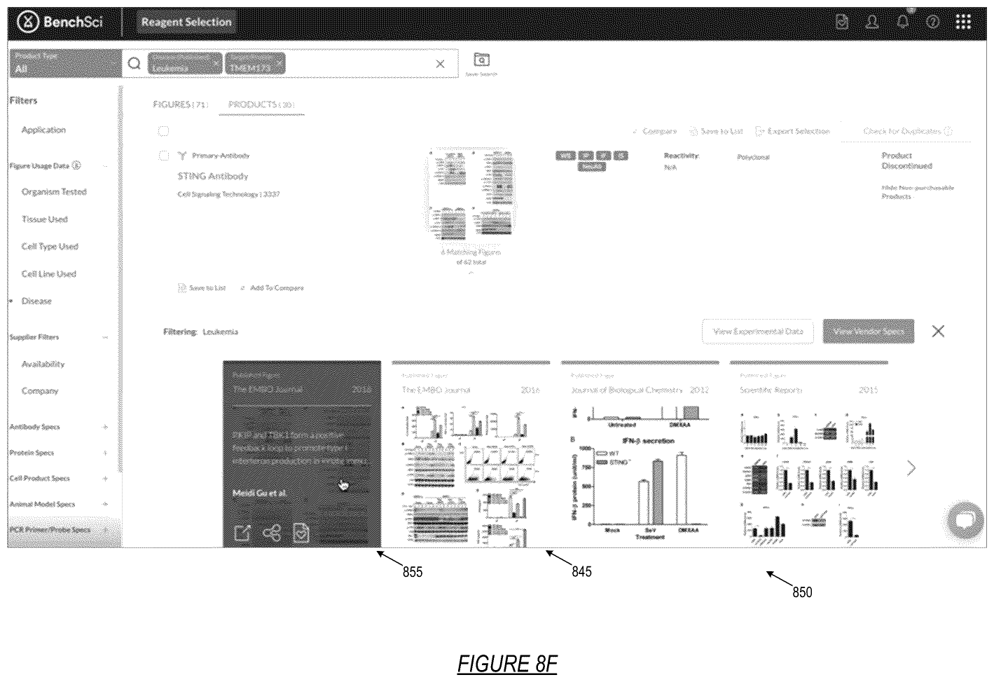

Turning to FIG. 3A, the user interface (300) is displayed. The user interface (300) may be displayed on a user device in response to opening a uniform resource locator (URL) in a web browser on the user device. The user interface includes multiple user interface elements, including the type selection element (302) and the entity identification element (305). The type selection element (302) is used to identify the entity type of a search term to be added to the entity identification element (305).

Turning to FIG. 3B, the user interface (308) is displayed as an update from the user interface (300) of FIG. 3A. The user interface (308) is updated to show the dropdown element (310).

The dropdown element (310) is displayed in response to selection of the type selection element (302). The dropdown element (310) displays the types of entities that may be selected to search using the system. The types of entities include antibodies, protein reagents, cell products, animal models, polymerase chain reaction (PCR) primers/probes, guide ribonucleic acid (gRNA), Cas Nuclease, RNA interference (RNAi) reagents, etc. Multiple types may be selected.

Turning to FIG. 3C, the user interface (320) is displayed as an update from the user interface (308) of FIG. 3B. The user interface (320) is updated to show the dropdown element (322).

The dropdown element (322) is displayed after the entity identification element (305) is selected. Text may be entered into the dropdown element (322) and the entered text may filter the list of items displayed in the dropdown element (322).

Turning to FIG. 4A, the user interface (400) is displayed as an update from the user interface (308) of FIG. 3B. The user interface (400) is updated to show the filter view (402) and the figure view (405). The filter view (402) includes user interfaces elements used to adjust filters that may be used by the system.

The figure view (405) displays a set of figures (including the FIG. 408)) that are relevant to the set of search terms from the entity identification element (305). Each of the figures in the figure view (405) are images from biomedical sources that have been ingested by the system. Being displayed in the figure view (405) indicates that the biomedical source, from which the image of the figure was obtained, is a match to the search requested by the user.

Turning to FIG. 4B, the user interface (420) is displayed as an update from the user interface (400) of FIG. 4A. The user interface (420) is updated to show the overlay (422) on the FIG. 408) in the figure view (405).

The overlay (422) displays text that corresponds to the biomedical source from which the FIG. 408) was extracted. The overlay (422) includes the type of the source (“Published Figure”), the name of the source (“eLife”), the date the source was made available (“2020”), at least a portion of a sentence extracted from the biomedical source (“PARP1 inhibitors trigger . . . ”), and a list of the authors of the biomedical source (“Chiho Kim, . . . ”). The overlay (422) also includes icon elements for viewing the biomedical source, sharing the biomedical source (e.g., as an email), and selecting the biomedical source as one of a set of favorite sources.

In one embodiment, the overlay (422) is at least partially transparent over the FIG. 408). Transparent overly of the overlay (422) shows the FIG. 408) beneath the overlay (422). The overlay (422) may show the tags from the FIG. 408).

Turning to FIG. 5, the user interface (500) is displayed as an update from the user interface (420) of FIG. 4B. The user interface (500) is updated to show the source view (502). The source view (502) includes the figure window (505) and the information window (508).

The figure window (505) includes the image (506) and the text (507). The image (506) is the image from the biomedical source that includes evidence of an experiment that is relevant to the search request from the user. Below the image (506), multiple icon elements are presented to view the biomedical source of the figure (e.g., load and display the publication of the biomedical source in a viewing program), share the biomedical source, and identify the biomedical source as a favorite biomedical source. The text (507) is text that corresponds to the image (506). The text (507) may be a legend of the image (506) that is extracted from the biomedical source. In one embodiment, the text (507) may be a sentence generated from the image (506) that identifies the entities and relationships therebetween from the image (506).

The information window (508) displays information extracted from the biomedical source from which the image (506) was extracted. The information window (508) includes the name of the biomedical source (“eLife”), the date of publication (“2020”), the title of the article that forms the biomedical source (“PARP1 inhibitors trigger . . . ”), the authors of the biomedical source (“Chiho Kim Et Al.”), etc.

The information window (508) also includes user interface elements that identify the entities and related products that the system identified within the biomedical source when ingesting the biomedical source.

The search tab element (510) is a user interface element that identifies the number of entities within the biomedical source that match to the search request. In one embodiment, selection of the search tab element (510) may automatically update the information window (508) to display the elements referenced by the search tab element (510). The information window (508) may be updated by scrolling to show sections for the tab elements (e.g., the tab elements identified as “Matching Your Search (2)”, “Antibodies (2)”, “Cell Products (2)”, and “RNAi (1)”). The elements referenced by the search tab element (510) include the item element (512) and the item element (513).

The item element (512) displays information about a product identified in the biomedical source. The item element (512) identifies the type of entity corresponding to the product (“Verified Antibody”), the name of the product (“STING (D2P2F) Rabbit mAb”), etc. The item element (512) also displays icon elements for sharing and liking (i.e., identifying as a favorite) the biomedical product identified in the item element (512). The item element (513) displays similar information but does not include the icon elements since the mouse is not hovering over the item element (513).

Turning to FIG. 6A, the specification view (602) is displayed in the user interface (600). The specification view (602) displays information, identified by the system, about a product (e.g., the product “STING (D2P2F) Rabbit mAb”). The specification view (602) displays several types of information that are accessible using the tabs titled “SPECS”, “FIGURES (311)”, “EXPERIMENTAL DATA”, “DUPLICATES”, and “TARGET INFO”. The user interface (600) shows a portion of the specification view (602) with information about the target, host, and clonality of the product.

Turning to FIG. 6B, the user interface (610) is displayed as an update from the user interface (600) of FIG. 6A. The user interface (610) is updated to show additional information, including information about the target, host, clonality, clone identification, conjugation, and verification of the product.

Turning to FIG. 6C, the user interface (620) is displayed as an update from the user interface (610) of FIG. 6B and in response to selecting the tab element (622). The update may also be displayed in response to scrolling down from the user interface (610) to the user interface (620). The user interface (620) shows the product figure view (623).

The product figure view (623) includes multiple figures from multiple biomedical sources. The multiple figures each correspond to the product described in the specification view (602), i.e., the product “STING (D2P2F) Rabbit mAb”. The images in the product figure view (623) may be filtered using the product filter view (625).

The product filter view (625) displays a list of filters. The filters limit the results presented in the product figure view (623).

Turning to FIG. 6D, the user interface (630) is displayed as an update from the user interface (620) of FIG. 6C and in response to scrolling down from the user interface (620). The update may also be displayed in response to selecting the “EXPERIMENTAL DATA” tab element (632). The user interface (630) shows the application view (635).

The application view (635) displays information about the applications of the product (“STING (D2P2F) Rabbit mAb”) from the biomedical sources ingested by the system. The product was used in figures depicting a “Western Blot” “256” times, figures depicting “Immunoprecipitation” “52” times, and in figures depicting “Immunostaining” “28” times.

Turning to FIG. 6E, the user interface (640) is displayed as an update from the user interface (630) of FIG. 6D and in response to scrolling down from the user interface (630). The user interface (640) shows the reactivity view (645).

The reactivity view (645) displays information about the reactivity of the product (“STING (D2P2F) Rabbit mAb”) from the biomedical sources ingested by the system. The product was shown in figures with the species as “Human” “157” times, figures with the species as “Mouse” “43” times, and figures with the species as “Herpes Simplex Virus (hsv)” “14” times.

Turning to FIG. 6F, the user interface (650) is displayed as an update from the user interface (640) of FIG. 6E and in response to scrolling down from the user interface (640). The user interface (650) shows additional experimental data about the product (“STING (D2P2F) Rabbit mAb”) from the biomedical sources ingested by the system. The additional experimental data identifies tissues used, cell types used, and cell lines used.

Turning to FIG. 6G, the user interface (660) is displayed as an update from the user interface (650) of FIG. 6F and in response to scrolling down from the user interface (650). The update may also be displayed in response to selecting the “DUPLICATES” tab element (652). The user interface (660) shows the duplicates view (655).

The duplicates view (655) identifies sets of multiple products that may be the same product. In one embodiment, the duplication between products may be identified using the names of the products. The duplicates view (655) indicates the products “STING (D2P2F) Rabbit mAb” and “STING (D2P2F) Rabbit mAb (BSA and Azide Free)” may be duplicates of each other.

Turning to FIG. 7A, the user interface (700) is displayed as an update from the user interface (420) of FIG. 4B and in response to selecting the “PRODUCTS” tab element (702). The user interface (700) shows the product view (705) and the filter view (710).

The product view (705) displays a set of rows with one row for each product relevant to the search requested by the user. The set of rows includes the row (708).

The row (708) displays information about a product. The information includes the entity type (“Verified Antibody”), the entity name (“STING (D2P2F) Rabbit mAb”), a figure from a biomedical source, and reactivity data.

The filter view (710) displays several filters that are available. Multiple types of filters are available, including filters for the type of organism tested, tissue used, cell type used, cell line used, disease. The filters further include filters for the suppliers of the products and filters based on the type of entity. The entity filters include antibody filters, protein filters, cell product filters, animal model filters, PCR primer filters, gRNA filters, cas nuclease filters, RNAi filters, etc.

Turning to FIG. 7B, the user interface (712) is displayed as an update from the user interface (700) of FIG. 7A and the antibody filter from the filter view (712).

The filter view (710) displays an expansion to the antibody filters. The expansion displays a set of categories that may be used to filter the results presented in the product view (705). The categories for antibodies include “Verification”, “Reactivity”, “Host”, “Isotype”, “Immunogen”, “Clonality”, “Clone ID”, etc.

Turning to FIG. 7C, the user interface (720) is displayed as an update from the user interface (712) of FIG. 7B. The user interface (720) is displayed in response to selecting the “Verification” category. The user interface (720) displays the verification view (722).

The verification view (722) displays elements for selecting the types of verification that are used to filter the results of the search request. Selecting a type of verification (e.g., “Overexpression”) may remove products from the search results that do not include biomedical sources linking the product to verification by overexpression. The row for “Overexpression” identifies the number of products (“20”) linked to verification by overexpression and identifies the number of products (“4”) that also include figures presenting evidence of overexpression. The search element at the top of the verification view (722) is used to search for the different types of verification. For example, searching for “e” may remove the “Orthogonal” type of verification since “Orthogonal” does not include the letter “e”.

Turning to FIG. 7D, the user interface (730) is displayed as an update from the user interface (720) of FIG. 7C. The user interface (730) is displayed in response to selecting to apply the filter for verification by overexpression from the user interface (720) of FIG. 7C. The search element (732) is updated to show the additional search term and the results in the product view (705) are updated to show results in accordance with the search request.

Turning to FIG. 7E, the user interface (740) is displayed as an update from the user interface (730) of FIG. 7D. The user interface (740) is displayed in response to selecting the “Availability” category from the “Supplier” filters on the filter view (710). The availability view (742) is used to select between displaying results for all products (including products that are not commercially available) and product that are commercially available.

Turning to FIG. 7F, the user interface (750) is displayed as an update from the user interface (740) of FIG. 7E. The user interface (750) is displayed in response to selecting the “Company” category from the “Supplier” filters on the filter view (710). The company view (752) is used to filter the results of a search based on the companies the produce the product. The company view (752) displays a set of rows, including the row (755). The row (755) is used to filter for products from the company “GeneCopoeia”. The row (755) indicates that “444” products from “GeneCopoeia” are presently in the search but that there are “0” biomedical sources with figures of relating the product to the company.

Turning to FIG. 8A, the user interface (800) is displayed. The user interface (800) is updated to reset the search to “TMEM173”, shown in the search element (801). The user interface (800) is further updated to display the figure view (802) in response to selecting the figure tab element (805).

Turning to FIG. 8B, the user interface (810) is displayed as an update from the user interface (800) of FIG. 8A. The user interface (810) is displayed in response to selecting the “Disease” category the filter view (710). The disease view (812) is used to filter the results of a search based on the type of disease. The disease view (812) displays a set of rows, including the row (815). The row (815) is used to filter for products used in relation to “Leukemia”. The row (815) indicates that “56” figures have been published in biomedical sources and the “15” of those figures are from supplier information about the product.

Turning to FIG. 8C, the user interface (820) is displayed as an update from the user interface (810) of FIG. 8B. The user interface (820) is displayed in response to selecting to apply the filter for leukemia from the user interface (810) of FIG. 8B. The search element (822) is updated to show the additional search term and the results in the figure view (802) are updated to show results in accordance with the search request.

Turning to FIG. 8D, the user interface (830) is displayed as an update from the user interface (820) of FIG. 8C. The user interface (830) is displayed in response to selecting the “PRODUCTS” tab element (832). The user interface (830) shows the updated product view (835) that includes results from the updated search request.

The product view (835) includes the row (837), which includes the figure element (838). The figure element (838) is one of multiple figures from multiple biomedical sources that include the product (“STING Antibody”) of the row (837) and accord with the parameters of the search. The figure element (838) includes the interface element (839). The interface element (839) includes text that indicates “62” figures, from the database of biomedical sources, are relevant to the product (“STING Antibody”) and the “6” of the “62” figures match with the search request.

Turning to FIG. 8E, the user interface (840) is displayed as an update from the user interface (830) of FIG. 8D. The user interface (840) is displayed in response to selecting the interface element (839). The user interface (840) is updated to display the product figure view (842).

The product figure view (842) displays four of the six figures identified by the interface element (839) as matching the parameters of the search request. The parameters are for a biomedical source to include two entities with a first entity having an entity type of “disease” and an entity name of “leukemia” and a second entity having an entity type of “Target/Protein” and an entity name of “TMEM173”. The product figure view (842) includes display of the figure element (845), which includes an overlay responsive to the mouse hovering over the figure element (845). The overlay to the figure element (845) presents additional information about the biomedical source from which the image of the figure element (845) was extracted.

Turning to FIG. 8F, the user interface (850) is displayed as an update from the user interface (840) of FIG. 8E. The user interface (850) is updated to display an overlay on the figure element (855) and remove the overlay from the figure element (845). The changing of the overlays of the figure element (845) and the figure element (855) is in response to moving the mouse cursor from hovering over the figure element (845) to hover over the figure element (855). The overlay to the figure element (855) presents additional information about the biomedical source from which the image of the figure element (855) was extracted. The figure element (855) and the figure element (845) include images extracted from the same biomedical source, i.e., from the source named “The EMBO Journal” published in “2016” by the authors “Meidi Gu et al.”.

Turning to FIG. 9A, the user interface (900) is displayed as an update from the user interface (850) of FIG. 8F. The user interface (900) is displayed in response to selecting the figure element (855) of FIG. 8F. The user interface (900) is updated to display the source view (902). The source view (902) includes the source figure view (905) and the source information view (908).

The source figure view (905) displays an image from the biomedical source. The image of the source figure view (905) is the same image displayed in the figure element (855) of FIG. 8F. The source figure view (905) also displays the legend text for the image from the biomedical source and displays icon elements for viewing the biomedical source, sharing the biomedical source, and liking the biomedical source.

The source information view (908) presents additional information about the biomedical source from which the image in the source figure view (905) was extracted. The source information view (908) includes the information element (910), the products element (912), and the more figures element (915).

The information element (910) presents additional information about the biomedical source. The information element (910) displays text that identifies the name of the publication of the biomedical source (“The EMBO Journal”), the date of the biomedical source (“2016”), the title of the article of the biomedical source (“RKIP and TBK1 form a positive feedback loop . . . ”). The information element (910) includes a view element that, when selected, presents the biomedical source in a viewer application.

The products element (912) presents information about products that are identified in the biomedical source. The products element (912) displays the product “STING Antibody” as a product that the system identified from within the biomedical source. When identified within the biomedical source, the product may be used in a biomedical experiment described in the biomedical source.

The figures element (915) displays additional figures from the biomedical source. The images of the figures within the figures element (915) are scaled down to fit within the source information view (908).

Turning to FIG. 9B, the user interface (950) is displayed as an update from the user interface (900) of FIG. 9A. The user interface (950) is displayed in response to scrolling the source information view (908). Scrolling the source information view (908) reveals additional figures from the biomedical source. Selecting one of the additional figures from the source information view (908) will load the selected figure to the source figure view (905) and update the legend text in the source figure view (905).

Turning to FIG. 10A, the user interface (1000) is displayed as an update from the user interface (820) of FIG. 8C. The user interface (1000) is displayed in response to selecting the sorting element (1002).

The sorting element (1002) is used to select between different sorting methods (i.e., algorithms) for the figures displayed in the figure view (1005). The sorting methods include “BEST MATCH”, “PUBLICATION DATE”, and “IMPACT FACTOR”. The best match method may rank figures based on the number of matching terms associated with the figure. The publication date method may sort the figures based on the dates of publication of the biomedical sources from which the figures were extracted. The impact factor method may sort the figures based on the number of citations to the biomedical source from which the figure was extracted. Selecting a different sorting method may update the order of the figures in the figure view (1005).

Turning to FIG. 10B, the user interface (1050) is displayed as an update from the user interface (1000) of FIG. 10A. The user interface (1050) is displayed in response to interaction with the source element (1052).

The source element (1052) is used to select between different types of sources of the biomedical sources that are used by the system to search for results to the search request. The types of sources include “Published”, “Supplier”, and “Third Party”. In the user interface (1050), the “Published” and “Third Party” sources are unselected leaving the “Supplier” sources, which are provided by the suppliers of the biomedical products identified by the system. The figure view (1005) is updated to show figures from biomedical sources that are provided by a supplier. With the source element (1052), biomedical sources from suppliers may be excluded from the results of the search request, which may improve the quality of the results by reducing duplicative figures.

Turning to FIG. 11A, the user interface (1100) is displayed. The user interface (1100) is displayed in response to creating a new search request based on a genetic sequence. The “Sequence” category in the “RNAi Specs” element is revealed in the filter view (1102).

Turning to FIG. 11B, the user interface (1110) is displayed. The user interface (1110) is displayed in response to selection of the “Sequence” category from the filter view (1102). Selection of the filter view (1102) triggers display of the sequence selection element (1112). The sequence selection element (1112) is used to filter the results of the search request to biomedical sources that include a known RNAi sequence.

Turning to FIG. 11C, the user interface (1120) is displayed. The user interface (1120) is displayed in response to interaction with the sequence selection element (1112). The user interface (1120) displays the product view (1125). The product view (1125) is updated with results for the search request that include genetic sequences, e.g., the sequence “TCTCTTGAA” from the row (1128).

Turning to FIG. 11D, the user interface (1130) is displayed. The user interface (1130) is displayed in response to selection of the sequence “TCTCTTGAA” of the row (1128) from the product view (1125). The user interface (1130) displays the sequence view (1135). The sequence view (1135) displays information about the sequence, including the targets for the sequence.

Turning to FIG. 11E, the user interface (1140) is displayed as an update to the user interface (1130) from FIG. 11D. The user interface (1140) is updated by scrolling down the sequence view (1135) to present multiple figures. The figures are images from biomedical sources that include the sequence (“TCTCTTGAA”) of the sequence view (1135).

Turning to FIG. 12A, the user interface (1200) is displayed. The user interface (1200) presents the product view (1202) with results for the search request identified in the search element (1205). The product view (1202) includes multiple rows that may be selected for a comparison of multiple products upon selection of the comparison element (1208).

Turning to FIG. 12B, the user interface (1220) is displayed as an update to the user interface (1200) of FIG. 12A. The user interface (1220) presents the detail comparison view (1225). The detail comparison view (1225) includes rows for each of the rows selected from the product view (1202) of FIG. 12A.

The detail comparison view (1225) presents several columns of information for the selected products. Included are columns for product identification information (e.g., a product name), supplier identification information (e.g., a supplier name), product catalog number information, figure statistics information (identifying, e.g., the number of figures analyzed by the system that include the product), application information, reactivity information, host information, clonality information, clone identification information, and conjugation information. The detail comparison view (1225) includes “duplicate” tags that identify rows that may be for duplicate products.

Turning to FIG. 12C, the user interface (1240) is displayed as an update to the user interface (1220) of FIG. 12B. The user interface (1240) presents the figure comparison view (1245). The figure comparison view (1245) displays figures for the products of the rows selected from the product view (1202) of FIG. 12A. The figure comparison view (1245) may be displayed in response to selection of the figure compare tab (1248). The figure comparison view (1245) includes “duplicate” tags that identify figures that may be for duplicate products.

Embodiments of the invention may be implemented on a computing system. Any combination of a mobile, a desktop, a server, a router, a switch, an embedded device, or other types of hardware may be used. For example, as shown in FIG. 13A, the computing system (1300) may include one or more computer processor(s) (1302), non-persistent storage (1304) (e.g., volatile memory, such as a random access memory (RAM), cache memory), persistent storage (1306) (e.g., a hard disk, an optical drive such as a compact disk (CD) drive or a digital versatile disk (DVD) drive, a flash memory, etc.), a communication interface (1312) (e.g., Bluetooth interface, infrared interface, network interface, optical interface, etc.), and numerous other elements and functionalities.

The computer processor(s) (1302) may be an integrated circuit for processing instructions. For example, the computer processor(s) (1302) may be one or more cores or micro-cores of a processor. The computing system (1300) may also include one or more input device(s) (1310), such as a touchscreen, a keyboard, a mouse, a microphone, a touchpad, an electronic pen, or any other type of input device.

The communication interface (1312) may include an integrated circuit for connecting the computing system (1300) to a network (not shown) (e.g., a local area network (LAN), a wide area network (WAN) such as the Internet, a mobile network, or any other type of network) and/or to another device, such as another computing device.

Further, the computing system (1300) may include one or more output device(s) (1308), such as a screen (e.g., a liquid crystal display (LCD), a plasma display, a touchscreen, a cathode ray tube (CRT) monitor, a projector, or other display device), a printer, an external storage, or any other output device. One or more of the output device(s) (1308) may be the same or different from the input device(s) (1310). The input and output device(s) (1310) and (1308) may be locally or remotely connected to the computer processor(s) (1302), non-persistent storage (1304), and persistent storage (1306). Many different types of computing systems exist, and the aforementioned input and output device(s) (1310) and (1308) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (1300) in FIG. 13A may be connected to or be a part of a network. For example, as shown in FIG. 13B, the network (1320) may include multiple nodes (e.g., node X (1322), node Y (1324)). Each node may correspond to a computing system, such as the computing system (1300) shown in FIG. 13A, or a group of nodes combined may correspond to the computing system (1300) shown in FIG. 13A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (1300) may be located at a remote location and connected to the other elements over a network.

Although not shown in FIG. 13B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (1322), node Y (1324)) in the network (1320) may be configured to provide services for a client device (1326). For example, the nodes may be part of a cloud computing system. The nodes may include functionality to receive requests from the client device (1326) and transmit responses to the client device (1326). The client device (1326) may be a computing system, such as the computing system (1300) shown in FIG. 13A. Further, the client device (1326) may include and/or perform all or a portion of one or more embodiments of the invention.

The computing system (1300) or group of computing systems described in FIGS. 13A and 13B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (1300) in FIG. 13A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (1300) of FIG. 13A, while performing one or more embodiments of the invention, may perform data comparison. Data comparison may be used to compare two or more data values (e.g., A, B). For example, one or more embodiments may determine whether A>B, A=B, A !=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (1300) in FIG. 13A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (1300) of FIG. 13A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (1300) of FIG. 13A and the nodes (e.g., node X (1322), node Y (1324)) and/or client device (1326) in FIG. 13B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
receiving a search request comprising an entity type and an entity name;
searching biomedical data to identify a set of biomedical sources that correspond to the entity name using an ontology library; and
presenting an image from a biomedical source, of the set of biomedical sources, wherein the image corresponds to the entity name.

2. The method of claim 1, further comprising:
presenting the image in a figure view with a set of images corresponding to the set of biomedical sources.

3. The method of claim 1, further comprising:
presenting the image in a product view, wherein the product view comprises a row of a set of rows, wherein the row comprises the image and product information in a plurality of fields.

4. The method of claim 1, further comprising:
ingesting a plurality of biomedical sources of the biomedical data to generate the ontology library,
wherein the plurality of biomedical sources comprises the set of biomedical sources,
wherein the ontology library defines a plurality of entitles using a plurality of entity types and a plurality of entity names,
wherein the plurality of entity types comprises the entity type,
wherein the plurality of entity names comprises the entity name, and
wherein the plurality of biomedical sources comprises the set of biomedical sources.

5. The method of claim 1, further comprising:
prior to receiving the search request, processing the biomedical source to record a correspondence between the entity type, the entity name, and the biomedical source.

6. The method of claim 1, further comprising:
prior to receiving the search request, processing at least a portion of the image with a machine learning model to identify a type of an entity, defined the ontology library, corresponding to the at least a portion of the image.

7. The method of claim 1, further comprising:
prior to receiving the search request, processing text from the biomedical source with a machine learning model to identify a type of an entity, defined in the ontology library, corresponding to the text.

8. The method of claim 1, further comprising:
presenting a plurality of images, corresponding to a plurality of biomedical sources, ordered using one of a plurality of criteria,
wherein a match criterion, of the plurality of criteria, orders the plurality of images using a number of matches of the entity name to text from the biomedical source,
wherein a date criterion, of the plurality of criteria, orders the plurality of images using a date of the biomedical source, and
wherein an impact criterion, of the plurality of criteria, orders the plurality of images using a number of citations to the biomedical source.

9. The method of claim 1, further comprising:
receiving the search request in response to selection of the entity name presented in a category presented in an entity type presented in a filter view; and
updating a set of search results, comprising the image, using the selection.

10. The method of claim 1, further comprising:
presenting a specification view of a product specification in response to selection of a product identifier, wherein the specification view presents detail information about a biomedical product; and
presenting a source view of a biomedical source.

11. A system comprising:
an information controller configured to receive a search request;
a result controller configured to search biomedical data; and
an application executing on one or more processors and configured for:
receiving, by the information controller, the search request comprising an entity type and an entity name;
searching, by the result controller, the biomedical data to identify a set of biomedical sources that correspond to the entity name using an ontology library; and
presenting an image from a biomedical source, of the set of biomedical sources, wherein the image corresponds to the entity name.

12. The system of claim 11, wherein the application is further configured for:
presenting the image in a figure view with a set of images corresponding to the set of biomedical sources.

13. The system of claim 11, wherein the application is further configured for:
presenting the image in a product view, wherein the product view comprises a row of a set of rows, wherein the row comprises the image and product information in a plurality of fields.

14. The system of claim 11, wherein the application is further configured for:
ingesting a plurality of biomedical sources of the biomedical data to generate the ontology library,
wherein the plurality of biomedical sources comprises the set of biomedical sources,
wherein the ontology library defines a plurality of entitles using a plurality of entity types and a plurality of entity names,
wherein the plurality of entity types comprises the entity type,
wherein the plurality of entity names comprises the entity name, and
wherein the plurality of biomedical sources comprises the set of biomedical sources.

15. The system of claim 11, wherein the application is further configured for:
prior to receiving the search request, processing the biomedical source to record a correspondence between the entity type, the entity name, and the biomedical source.

16. The system of claim 11, wherein the application is further configured for:
prior to receiving the search request, processing at least a portion of the image with a machine learning model to identify a type of an entity, defined the ontology library, corresponding to the at least a portion of the image.

17. The system of claim 11, wherein the application is further configured for:
prior to receiving the search request, processing text from the biomedical source with a machine learning model to identify a type of an entity, defined in the ontology library, corresponding to the text.

18. The system of claim 11, wherein the application is further configured for:
presenting a plurality of images, corresponding to a plurality of biomedical sources, ordered using one of a plurality of criteria,
wherein a match criterion, of the plurality of criteria, orders the plurality of images using a number of matches of the entity name to text from the biomedical source,
wherein a date criterion, of the plurality of criteria, orders the plurality of images using a date of the biomedical source, and
wherein an impact criterion, of the plurality of criteria, orders the plurality of images using a number of citations to the biomedical source.

19. The system of claim 11, wherein the application is further configured for:
receiving the search request in response to selection of the entity name presented in a category presented in an entity type presented in a filter view; and
updating a set of search results, comprising the image, using the selection.

20. A non-transitory computer-readable medium storing program instructions that, when executed by one or more processors, cause a computing system to perform operations comprising:
receiving a search request comprising an entity type and an entity name;
searching biomedical data to identify a set of biomedical sources that correspond to the entity name using an ontology library; and
presenting an image from a biomedical source, of the set of biomedical sources, wherein the image corresponds to the entity name.
